(19) United States
(12) Patent Application Publication
(10) Pub. No.: US 2018/0298099 A1
(43) Pub. Date: Oct. 18, 2018

(54) T-Cell Recepter (TCR) Targeting Therapies for Immune-Mediated Adverse Drug Reactions (ADRs) and Other Conditions

(71) Applicant: Harry C. Ledebur, JR., Kalamazoo, MI (US)

(72) Inventor: Harry C. Ledebur, JR., Kalamazoo, MI (US)

(21) Appl. No.: 15/759,095

(22) PCT Filed: Sep. 16, 2016
(86) PCT No.: PCT/US2016/052252

§ 371 Date: Mar. 9, 2018

Related U.S. Application Data
(60) Provisional application No. 62/219,411, filed on Sep. 16, 2015.
(60) Provisional application No. 62/268,781, filed on Dec. 17, 2015.

(51) Int. Cl. C07K 16/28; A61K 39/395; A61K 45/06; A61P 17/00

(52) U.S. Cl. CPC C07K 16/2809; A61K 39/39541; A61K 2039/545; A61P 17/00; A61K 45/06

(57) ABSTRACT

The present invention provides methods and compositions for treating immune mediated adverse drug reactions using anti-αβ TCR antibodies and fragments thereof. The adverse drug reactions treated according to the invention include severe cutaneous adverse reactions, idiosyncratic liver injury, and idiopathic drug-induced liver disease. The invention also provides methods and compositions for treating conditions such as epidermolysis bullosa, pemphigus vulgaris, cutaneous T cell lymphoma, and Goodpasture syndrome where T cells play a significant role. Anti-α TCR antibodies used in the treatment methods of the invention include T10B9, MEDI-500 and TOL101.

### CROSS REFERENCE TO RELATED APPLICATIONS

This application claims priority to U.S. Provisional Application No. 62/219,411, filed Sep. 16, 2015, and U.S. Provisional Application No. 62/268,781, filed Dec. 17, 2015, each of which is hereby incorporated by reference in its entirely.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: TCR_ADR_SeqList.txt, date recorded: Sep. 15, 2016, file size 18.9 kilobytes).

### FIELD

The present invention relates to antibodies and methods for selectively inhibiting (TCR+) T-cell immune responses. In particular, the present invention relates to treatment of immune-mediated adverse drug reactions (IM-ADR), e.g., idiopathic adverse drug reactions (ADRs), drug allergy, severe cutaneous adverse reactions (SCARs) (e.g., Stevens Johnson syndrome, acute generalised exanthematous pustulosis, drug reaction with eosinophilia and systemic symptoms), and certain other conditions in which T cells play a significant role e.g., pemphigus vulgaris, epidermolysis bullosa, and Goodpasture syndrome, using anti-αβ TCR antibodies and antibody fragments. In some embodiments, the anti-αβ TCR antibodies are T10B9, MEDI-500, TOL101, chimeric or humanized antibodies derived from T10B9, MEDI-500, or TOL101 or variants thereof or antibody fragments derived from T10B9, MEDI-500, or TOL101. In some embodiments, the methods for selectively inhibiting (TCR+) T-cell immune responses comprise administering antibodies, antibody fragments or molecules that recognize, bind or interact with the same TCR epitope as TOL101, T10B9 or MEDI-500.

### BACKGROUND

Immune-mediated adverse drug reactions (IM-ADR) can also be referred to as idiosyncratic drug reactions (IDR), Type B adverse drug reactions (ADRs), immune-related adverse drug reactions (irADRs), or drug allergies. In total, these reactions represent a significant cause of morbidity and mortality for patients. IM-ADRs can be systemic or focused on a single organ including skin, liver, pancreas, lung, and bone marrow. The term “IM-ADR” designates an adverse drug reaction that does not occur in most patients treated with a drug and is not predictable based on the on-target pharmacologic activity or pharmacologic principles of the drug. The reactions were originally considered idiopathic, but are now known to stem from off-target drug activity and are immune-mediated reactions. These reactions are not the most common type of adverse drug reaction, but they can be life threatening. Although the exact mechanism for an IM-ADR may differ from person to person and the drug, in general, drugs and drug-derived products interact with major histocompatibility complex (MHC) or T cell receptor to initiate the reaction. More details on current understanding of IDRs are provided in a review article by Uetrecht and Naisbitt (Pharmacol Rev 65:779-808, April 2013) and White et al., (J Allergy Clin Immunol 136(2):219-234, 2015), incorporated by reference herein in its entirety).

Immune-mediated ADRs encompass a number of distinct clinical diagnoses that comprise both (antibody-mediated, Gell-Coombs types I-III) and purely T cell-mediated (Gell-Coombs type IV) reactions (White et al., 2015). The clinically relevant T cell-mediated drug reactions have been classified into delayed exanthema without systemic symptoms (maculopapular eruption), contact dermatitis, drug-induced hypersensitivity syndrome/drug reaction with eosinophilia and systemic symptoms (DRESS)/hypersensitivity syndrome, Stevens-Johnson syndrome (SJS)/toxic epidermal necrolysis (TEN), acute generalized exanthematous pustulosis, fixed drug eruption, and single organ involvement pathologies, such as drug-induced liver injury and pancreatitis.

IDRs can also be classified into four major types: i) skin rashes (this includes cutaneous adverse drug reactions), ii) liver injury such as idiosyncratic liver injury (IDILI), iii) hematologic adverse reactions, and iv) drug-induced autoimmunity (Uetrecht and Naisbitt, 2013).

Cutaneous adverse drug reactions (cADR) are unpredictable and represent a plethora of skin diseases of varying degrees of severity (Bellón T., Curr. Immunol. Rev., 2014, 10, 24-32, incorporated by reference herein in its entirety). Those of most concern are usually referred to as severe cutaneous adverse reactions (SCARs), and include acute generalized exanthematous pustulosis (AGEP), drug reaction with cosinophilia and systemic symptoms (DRESS), also known as drug-induced hypersensitivity syndrome or hypersensitivity syndrome (DIHS/HSS), Stevens-Johnson's syndrome (SJS), and toxic epidermal necrolysis (TEN).

Stevens-Johnson syndrome (SJS) and toxic epidermal necrolysis (TEN) are a life threatening mucocutaneous reactions that are primarily drug-induced. SJS and TEN were once thought to be separate conditions, but are now considered part of a single disease continuum differing by the extent of skin detachment. SJS is characterized as the degree of epidermal detachment involving less than 10%/o of body surface area (BSA), while TEN is defined by the detachment greater than 30% of BSA. The cases with severity in-between (10-30%) are diagnosed as SJS/TEN overlap. The mortality rate for SJS/TEN can be as high as 30%. SJS/TEN is a delayed-type hypersensitivity reaction with a typical latency period of 4-28 days. The major theory to explain epidermal detachment of SJS/TEN is CD8+ T cell-mediated and NK-cell mediated cytotoxicity. Histopathology of SJS/TEN skin biopsy manifests extensive keratinocyte apoptosis and massive epidermal necrosis and detachment with a sparse dermal monocytic (predominantly T-cell) infiltrate (Pan et al., Curr. Immunol. Rev., 2014, 10, 51-61; Dodiuk-Gad et al., Am J Clin Dermatol, 2015, 16:475-493, incorporated by reference herein in its entirety).

DRESS is an IM-ADR also known as drug induced hypersensitivity syndrome (DIHS). DRESS has a later onset and longer duration than other drug reactions. The clinical manifestations of DRESS/DIHS include cutaneous eruption with fever, lymphadenopathy, hematologic abnormalities, and multiorgan involvement. Although the precise pathogenesis of DRESS is not fully understood, all evidence suggests that DRESS is a T-cell-mediated hypersensitivity reaction to drugs (Bellón T., Curr. Immunol. Rev., 2014, 10, 24-32)

The common culprit drugs for SCARs include aromatic antiepileptics (e.g., carbamazepine, phenytoin, lamotrigine, phenobarbital), antibiotics (e.g., sulfamethoxazole, sulfonamides, abacavir, nevirapine, dapsone), NSAIDs, and allopurinol, etc. Although rare, SCARs have significant public health impact because of the unpredictability, high mortality and morbidity (Dodiuk-Gad et al., Am J Clin Dermatol, 2015, 16:475-493).

Studies show that SCARs and T-cell mediated IM-ADRs are delayed type IV hypersensitivity reactions in which a T cell-mediated drug-specific immune response is responsible for triggering the disease (Bellón T., Curr. Immunol. Rev., 2014, 10, 24-32; White et al., 2015). Specific T cell subpopulations develop in response to certain environments and produce cytokines that orchestrate the various phenotypes. Tc1, Th1, Th2, Th17, and Treg, among other T cell subpopulations, participate in several SCARs and IM-ADRs. Immune cells belonging to the system, classically known as the innate immune system, comprising natural killer cells, monocytes, macrophages and dendritic cells, can also participate in shaping specific immune responses in various clinical entities. Additionally, tissue resident cells including keratinocytes can contribute to epidermal damage by secreting chemokines that attract proinflammatory immunocytes. The final phenotypes in each clinical entity result from the interactions between the cell types and their products (Bellón T., Curr. Immunol. Rev., 2014, 10, 24-32).

IM-ADRs, SCARs, the role of T cells and other immune cells in SCARs, animal models for studying SCARs and treatment for SCARs have been described in various articles including Bellón T., Curr. Immunol. Rev., 2014, 10, 24-32; Pan et al., Curr. Immunol. Rev., 2014, 10, 51-61 Azukizawa H., Curr. Immunol. Rev., 2014, 10, 19-23; Valeyrie-Allanore L. and Roujeau J C., Curr. Immunol. Rev., 2014, 10, 1-3; Pichler W. and Watkins S., Curr. Immunol. Rev., 2014, 10, 7-18: Stern R. and Mockenhaupt M., Curr. Immunol. Rev., 2014, 10, 4-6; Su S C and Wen-Hung C., Curr. Immunol. Rev., 2014, 10, 33-40: Shiohara T. et al., Curr. Immunol. Rev., 2014, 10, 41-50; and Aihara M. et al., J. of Dermatol., 2015, 42, 768-777, White et al., 2015, all of which are incorporated by reference herein in their entirety.

T cells have also been implicated in other cutaneous conditions such as epidermolysis bullosa and pemphigus vulgaris. Pemphigus vulgaris (PV) is a life-threatening autoimmune blistering disease caused by anti-desmoglein (Dsg) IgG that leads to a loss of epidermal cell-cell adhesion, called acantholysis, leading to chronic, progressive blistering of the mucous membranes and skin. Although the production of pathogenic antibodies is key to the development of PV, recent studies show that T cells play an important role in the pathogenesis of PV (Amber K. et al., Exp. Dermatol., 2013, 22, 699-704; incorporated by reference herein in its entirety). Epidermolysis bullosa (EB) is a group of inherited disorders that involves the formation of blisters following minor trauma. An autoimmune form of EB is known as epidermolysis bullosa acquisita (EBA) and is characterized by subepidermal blisters and autoantibodies against type VII collagen, the main constituent of the anchoring fibrils at the dermal-epidermal junction (DEJ). Studies show that T cells are required for the production of autoantibodies in the experimental model of EBA (Sitaru A. et al., J. of Immunol., 2010, 184, 1596-1603, incorporated by reference herein in its entirety). Another condition in which T cells play a role in the pathogenesis is Goodpasture's Syndrome, also known as anti-glomerular basement membrane disease (anti-GBM-disease), where patients develop autoantibodies against the type IV collagen protein, α3(IV)NC1, which is expressed in the glomerular basement membrane, alveolar basement membrane, and basement membranes in the testis, inner ear, eye and the choroid plexus. Genetic studies have revealed a strong link between anti-GBM disease and HLA-DRB1*1501 and DRB1*1502 implicating the role of T cells (Hellmark and Segelmark, “Diagnosis and classification of Goodpastures disease (anti-GBM),” 2014, Journal of Autoimmunity, (48-49), 108-112). Although T cells have been shown to play an important role in these conditions, T cell modulating therapies have not yet been explored for these conditions.

Current treatment for IM-ADRs and SCARs includes withdrawal of the offending drug accompanied by supportive treatment depending on severity and availability in a burns unit (White et al., 2015; Suran L. Fernando (2013); Severe Cutaneous Adverse Reactions, Skin Biopsy—Diagnosis and Treatment, Prof. Suran Fernando (Ed.), ISBN: 978-953-51-1173-3, InTech, DOI: 10.5772/54820. Available from: http://www.intechopen.com/books/skin-biopsy-diagnosis-and-treatment/severe-cutaneous-adverse-reaction; incorporated by reference herein in its entirety). For instance, supportive treatment of severe SJS/TEN depends on severity and availability in a burns unit to address the manifestations and complications of acute skin failure including monitoring of fluid-electrolyte balance, provision of enteral or parenteral nutrition, wound care and treatment of sepsis (Fernando, 2013). In addition, supportive care of affected mucosal surfaces is required. This includes aggressive ocular lubrication, topical corticosteroids and topical antibiotics, hygienic mouthwashes and topical oral anesthetics, and monitoring for urinary retention (Fernando, 2013). A number of potential treatment regimens and drugs have been evaluated in human clinical study, but none have definitively been proven to be an effective in treatment for SJS/TEN. Moreover, no optimal treatment regimen has been established and there are no internationally accepted management guidelines for these diseases (White et al., 2015).

Thus, there exists a tremendous unmet medical need for an agent capable of treating a wide spectrum of IM-ADRs and other conditions mediated by T cells.

### SUMMARY

Unless otherwise defined, all technical and scientific terms used herein have the same meaning as commonly understood by one of ordinary skill in the art to which this present technology belongs. Although methods and materials similar or equivalent to those described herein can be used in the practice or testing of the present technology, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

Although it has been shown that T cells play an important role in immune-mediated adverse drug reactions (e.g. IDILI, SJS, TEN, AGEP, DRESS) and other conditions such as epidermolysis bullosa, pemphigus vulgaris, and Goodpasture syndrome, exploration of T-cell specific therapies for these conditions has been limited (White et al., 2015). One of the reasons for lack of interest in exploring T-cell specific therapies is because T-cell specific therapeutic agents, such as monoclonal antibodies, are likely to be mitogenic and elicit the release of cytokines and other inflammatory mediators such as granzyme and perforin. For example, OKT3 (muromonab), a monoclonal antibody that binds to the CD3 subunit of the TCR, is a potent mitogen and stimulates the release of high amounts of cytokines (cytokine release storm). Stimulation of T cells and release of cytokines and other inflammatory mediators induced by T-cell specific agents would exacerbate the disease and could even be lethal to the subject. A second concern with the use of T-cell specific agents for treating the above conditions is a prolonged/long-term depletion of T cells which would increase the risk of opportunistic infections. For instance, polyclonal antisera against T-lymphocytes such as rabbit antilymphocyte globulin (Thymoglobulin) significantly suppresses T-cells for up to 90 days and has a profound B-cell inhibitory effect from 15 to 980 days. Similarly, campath, a CD52-specific monoclonal antibody that targets T and B lymphocytes profoundly suppresses T-cells for up to 330 days and B-cells for 90 to 150 days after an initial dose of 30 mg. Newer anti-CD3 mAbs, such as otelixizumab, also lead to prolonged periods of T cell depletion/suppression (e.g. at least 3 weeks or longer), and otelixizumab is also associated with a significant risk of reactivation of Epstein Barr virus (EBV) and cytokine release syndrome. IL-2R specific antibodies, such as basiliximab and daclizumab, target activated T cells without depleting them: however, due to their longer half-life, they suppress T cell function for more than six weeks (and more than 3 months with daclizumab). A prolonged depletion of T cells or a prolonged suppression of their function leads to opportunistic infection, a well known side effect of antibodies such as campath and otelixizumab. Patients suffering from adverse drug reactions are very susceptible to infections, particularly, secondary skin infections such as cellulitis, that can lead to life threatening complications, including sepsis. Therefore, depletion of T-cells or suppression of T-cell function for a long period of time is not very desirable for patients suffering from adverse drug reactions. Moreover, administration of prophylactic treatment to combat certain opportunistic infections, as done along with administration of campath for the treatment of CLL, is also not desirable in patients with adverse drug reactions, because any additional drug can also exacerbate the disease. Another concern with targeting T cells, in particular T-cell receptors, as a therapy for adverse drug reactions is that these therapies would also downregulate regulatory (Treg) cells. Treg cells are considered to play a major role in the immunopathology of adverse drug reactions (Shiohara T. et al., Curr. Immunol. Rev., 2014, 10, 41-50). Specifically, Treg cells suppress the function of effector T cells and limit the pathological damage induced by T effector cells. Depletion or suppression of Treg cells or their function by T-cell specific agents would exacerbate the conditions. A further concern with targeting T cells is the well-known toxicities for such agents. Cyclosporine is a potent T cell immunosuppressant, but also has considerable safety and toxicity concerns, including hypertension, hyperlipidemia, nephrotoxicity, hepatotoxicity, pancreatitis, peptic ulcers, thrombotic microangiopathy, opportunistic infection, neurotoxicity, tremor, gingival hyperplasia and hirsutism. Campath can lead to serious, sometimes fatal, hematologic complications and infections and antilymphocyte globulin has the potential for serious immune-mediated reactions, including cytokine release syndrome. Because of these concerns, one skilled in the art would not have expected anti-TCR antibodies to be effective in treating T-cell mediated conditions such as immune-mediated adverse drug reactions, idiosyncratic drug reactions, epidermolysis bullosa, pemphigus vulgaris, and Goodpasture syndrome. The methods and compositions provided by the present invention are, therefore, unexpected and surprising.

In various embodiments, the present invention provides methods and compositions for treating T cell mediated conditions using anti-αβ TCR antibodies or fragments thereof. T cell mediated conditions that can be treated according to the present invention include, but are not limited to, immune-mediated adverse drug reactions, drug allergy, Type B adverse drug reactions, idiosyncratic adverse drug reactions (IDRs) such as skin rashes including acute generalized exanthematous pustulosis (AGEP), drug reaction with eosinophilia and systemic symptoms (DRESS), Stevens-Johnson's syndrome (SJS), and toxic epidermal necrolysis (TEN), single organ pathologies like liver injuries such as idiosyncratic liver injury (IDILI) and other diseases such as epidermolysis bullosa, pemphigus vulgaris, cutaneous T cell lymphoma (CTCL), and Goodpasture Syndrome.

In some embodiments, the anti-αβ TCR antibodies used in the methods of the present invention are T10B9, MEDI-500, and TOL101. The invention also contemplates the use of chimeric, humanized, or other variants of T10B9, MEDI-500, and TOL101 as well as antibody fragments derived from T10B9, MEDI-500, and TOL101. The invention also encompasses antibodies, antibody fragments or molecules that recognize, bind or interact with the same TCR epitope as TOL101, T10B9 or MEDI-500.

In some embodiments, the methods for selectively inhibiting (TCR+) T-cell immune responses comprise administering antibodies, antibody fragments or molecules that recognize, bind or interact with the same TCR epitope as TOL101, T10B9 or MEDI-500.

TOL101 is described in International Appl. No. PCT/US 2012/063583, filed Nov. 5, 2012: the contents of which are incorporated by reference herein their entirety. TOL101 is produced by the TOL101 Master Cell Bank (MCB) hybridoma. The TOL101 MCB hybridoma was deposited on Nov. 2, 2012 at the American Type Culture Collection (ATCC), 10801 University Boulevard, Manassas, Va. 20110-2209, under the provisions of the Budapest Treaty on the International Recognition of the Deposit of Microorganisms for the Purpose of Patent Procedures, and assigned ATCC accession number PTA-13293.

In one aspect, the present invention provides a method for treating immune-mediated adverse drug reactions in a subject in need thereof comprising administering to the subject a therapeutically effective dose of T10B9, MEDI-500, TOL101 or a fragment thereof. Adverse drug reactions that may be treated according to the invention include SJS, TEN, AGEP, DRESS, and IDILI.

In another aspect, the present invention provides a method for treating severe cutaneous adverse reactions in a subject in need thereof comprising administering to the subject a therapeutically effective dose of T10B9, MEDI-500, TOL101 or a fragment thereof. Severe cutaneous adverse reactions that may be treated according to the invention include SJS, TEN, AGEP, and DRESS.

In yet another aspect, the present invention provides a method for treating a T cell mediated cutaneous condition in a subject in need thereof comprising administering to the subject a therapeutically effective dose of T10B9, MEDI-500, TOL101 or a fragment thereof, wherein the T cell mediated cutaneous condition is selected from the group consisting of epidermolysis bullosa, pemphigus vulgaris and cutaneous T cell lymphoma.

In yet another aspect, the present invention provides a method for treating an autoimmune disease in a subject in need thereof comprising administering to the subject a therapeutically effective dose of T10B9, MEDI-500, TOL101 or a fragment thereof, wherein the autoimmune disease is selected from the group consisting of pemphigus vulgaris, epidermolysis bullosa, and Goodpasture Syndrome.

In various embodiments, administration of the anti-αβ TCR antibody (e.g. T10B9, MEDI-500, and TOL101) or fragment thereof according to the methods of the invention provides transient and rapid removal of CD3+, CD8+ and/or CD4+ T cells from affected areas such as cutaneous adverse reaction sites. Transient and rapid removal of T cells can mitigate the progression of T-cell mediated conditions as well as morbidity and mortality associated with these diseases.

In one embodiment, administration of the anti-αβ TCR antibody or fragment thereof according to the methods of the invention does not induce production of a cytokine upon binding to the αβ TCR on a T cell. In a further embodiment, administration of the anti-αβ TCR antibody or fragment thereof according to the invention does not induce production of Tumor Necrosis Factor-alpha (TNF-α), Interferon-gamma (IFN-γ). Interleukin-2 and/or Interleukin-6 upon binding to the αβ TCR on a T cell. In another embodiment, the anti-αβ TCR antibody or fragment thereof does not produce levels of cytokines associated with cytokine release syndrome upon binding to the αβ TCR on a T cell. The cytokines associated with cytokine release syndrome include TNF-α, IFN-γ, Interleukin-2 and/or Interleukin-6. In another embodiment, administration of the anti-αβ TCR antibody or fragment thereof induces the production of less than 500 pg/mL of TNF-α, IFN-γ, IL-2, and/or IL-6 upon binding to αβ TCR on a T cell. In another embodiment, the anti-αβ TCR antibody or fragment thereof administered according to the invention binds to αβ TCR on a T cell, wherein the antibody binds to αβ TCR and reduces the surface expression of αβ TCR and CD3 on the T cell, and wherein the antibody does not deplete T cells. In another embodiment, the anti-αβ TCR antibody or fragment thereof induces phosphorylation of AKT or ERK upon binding the αβ TCR. In another embodiment, administration of the anti-αβ TCR antibody or fragment thereof induces the production, proliferation, and/or activity of regulatory T cells (Tregs) in the subject. In some embodiments, Tregs are phenotypically CD2+CD4+CD25+FOXP3+CD127lo Tregs.

In another embodiment, administration of the anti-αβ TCR antibody or fragment thereof induces calcium flux in less than 60% of αβ TCR+ T cells. In a further embodiment, the antibody or fragment thereof induces calcium flux in less than 50% of αβ TCR+ T cells. In a further embodiment, the antibody or fragment thereof induces calcium flux in less than 40% of αβ TCR+ T cells. In a further embodiment, the antibody or fragment thereof induces calcium flux in less than 30% of αβ TCR+ T cells. In a further embodiment, the antibody or fragment thereof induces calcium flux in less than 20% of αβ TCR+ T cells. In a further embodiment, the antibody or fragment thereof induces calcium flux in less than 15% of αβ TCR+ T cells. In a still further embodiment, the antibody or fragment thereof induces calcium flux in less than 10% of αβ TCR+ T cells.

In another embodiment, the anti-αβ TCR antibody or fragment thereof binds to αβ TCR on a T cell, wherein binding of the antibody or fragment to the TCR or cross-linking of the antibody or fragment does not induce proliferation of the T cell.

In some embodiments, the antibody fragment that may be administered to treat T cell mediated condition includes an scFv, an Fab fragment, an Fab′ fragment, and an F(ab)′ fragment.

In one embodiment, administration of the anti-αβ TCR antibody or fragment thereof according to the methods of the invention induces a Human anti-mouse antibody (HAMA) response in fewer than 20% of the human patients administered the antibody or fragment thereof as measured by Enzyme Linked Immunosorbent Assay (ELISA). In a further embodiment, the antibody or antigen binding fragment thereof induces a HAMA response in fewer than 10% or 5% of the human patients administered the antibody or fragment thereof as measured by ELISA.

In a further aspect, the methods of the invention comprise administering an anti-αβ TCR antibody or antibody fragment thereof in an amount from about 14 mg/day to about 52 mg/day to a subject in need thereof.

In one embodiment, the present invention provides a method for treating a T cell mediated cutaneous condition, comprising administering an αβ TCR antibody or fragment thereof in an amount from about 7 mg/day to about 58 mg/day to a subject in need thereof. In a further embodiment, the anti-αβ TCR antibody or fragment thereof is administered in an amount of 7 mg/day, 14 mg/day, 21 mg/day, 28 mg/day, 30 mg/day, 32 mg/day, 34 mg/day, 35 mg/day, 36 mg/day, 38 mg/day, 40 mg/day, 42 mg/day, 44 mg/day, 46 mg/day, 48 mg/day, 50 mg/day, 52 mg/day, 54 mg/day, 56 mg/day or 58 mg/day, or combinations thereof.

In one embodiment, the present invention provides a method for treating a T cell mediated cutaneous condition, comprising administering an αβ TCR antibody or fragment thereof in a dosing schedule comprising 14 mg at day 1, 21 mg at day 2, 28 mg at day 3, 42 mg at day 4, and 42 mg at day 5. In a further embodiment, the antibody or fragment thereof is administered in a dosing schedule comprising 14 mg at day 1, 21 mg at day 2, 28 mg at day 3, 42 mg at day 4, 42 mg at day 5, and 42 mg at day 6. In one embodiment, the αβ TCR antibody or antigen binding fragment thereof is administered once per day.

In one embodiment, the IM-ADR treated according to the methods of the invention is a drug allergy. In another embodiment, the IM-ADRs treated according to the methods of the invention include delayed exanthema without systemic symptoms (maculopapular eruption), contact dermatitis, drug-induced hypersensitivity syndrome/drug reaction with eosinophilia and systemic symptoms (DRESS)/hypersensitivity syndrome, Stevens-Johnson syndrome (SJS)/toxic epidermal necrolysis (TEN), acute generalized exanthematous pustulosis, fixed drug eruption, and single organ involvement pathologies, such as drug-induced liver injury and pancreatitis.

In some embodiments, the IM-ADR treated according to the methods of the invention is a T cell mediated hypersensitivity condition. The T cell mediated hypersensitivity conditions include, but are not limited to, delayed exanthema without systemic symptoms (maculopapular eruption), contact dermatitis, drug-induced hypersensitivity syndrome/drug reaction with eosinophilia and systemic symptoms (DRESS)/hypersensitivity syndrome, Stevens-Johnson syndrome (SJS)/toxic epidermal necrolysis (TEN), acute generalized exanthematous pustulosis, fixed drug eruption, and single organ involvement pathologies, such as drug-induced liver injury and pancreatitis.

In certain embodiments, the anti-αβ TCR antibody or antigen binding fragment administered according to the invention reduces CD3+ T cell counts to <25 T cell/mm³. In some embodiments, administration of the anti-αβ TCR antibody or antigen binding fragment thereof reduces CD3+ T cell counts to 50%, 75% or 90% of baseline.

In some embodiments, the anti-αβ TCR antibody or antigen binding fragment thereof allows for a CD3+ T cell rate of recovery of about 7, 10, 15, 20, 25 or 30 days. That is, CD3+ T cells that are depleted or rendered non-functional by administration of the anti-αβ TCR antibody or antigen binding fragment thereof are recovered by about 7, 10, 15, 20, 25 or 30 days after administration.

In one embodiment, administration of the anti-αβ TCR antibody or antigen binding fragment thereof does not induce an immunological response to the antibody or antigen-binding fragment itself. For instance, in one embodiment, the anti-αβ TCR antibody or antigen binding fragment thereof results in an anti-drug response of <20%.

In one embodiment, the anti-αβ TCR antibody or antigen binding fragment thereof permits once daily dosing. In some embodiments, the anti-αβ TCR antibody or antigen binding fragment thereof does not suppress or delete γδ T-cells, B cells, other white blood cells, or platelets.

In one embodiment, the anti-αβ TCR antibody or antigen binding fragment thereof administered according to the invention exhibits a reduced immune activation potential.

In one embodiment, the activity of the anti-αβ TCR antibody or antigen binding fragment thereof is mimicked by a chemical entity, peptide or RNA/DNA-based molecule. In some embodiments, the activity of TOL101, T10B9 or MEDI-500 is mimicked by a chemical entity, peptide or RNA/DNA-based molecule.

In some embodiments, the IM-ADR occurs subsequent to administration of one or more checkpoint inhibitors to the subject. In further embodiments, the checkpoint inhibitor is selected from ipilimumab, nivolumab, pembrolizumab, and atezolizumab, or a combination thereof. In some embodiments, the IM-ADR that occurs subsequent to administration of one or more checkpoint inhibitors is selected from a drug allergy. Type B adverse drug reaction, IDR. AGEP, DRESS, SJS, IDILI, epidermylosis bullosa, pemphigus vulgaris, cutaneous T cell lymphoma (CTCL), and Goodpasture Syndrome, or a combination thereof. In certain embodiments, the IM-ADR is SJS/TEN.

In some embodiments, the present disclosure provides methods for treating a subject comprising administering to the subject one or more checkpoint inhibitors and an anti-αβ T cell receptor (TCR) antibody or an antigen binding fragment thereof. In further embodiments, the one or more checkpoint inhibitors is selected from ipilimumab, nivolumab, pembrolizumab, and atezolizumab, or a combination thereof; and the anti-αβ TCR antibody or antigen binding fragment is selected from the group consisting of T10B9, MEDI-500, and TOL-101. In some embodiments, the one or more checkpoint inhibitors and the anti-αβ T cell receptor (TCR) antibody or an antigen binding fragment thereof are administered to the subject concurrently or sequentially. In some embodiments, the methods reduce the incidence or severity of, or prevent the onset of, an ADR associated with or caused by the one or more checkpoint inhibitors. In some embodiments, the ADR is selected from a drug allergy, Type B adverse drug reaction, IDR, AGEP, DRESS, SJS, IDILI, epidermolysis bullosa, pemphigus vulgaris, cutaneous T cell lymphoma (CTCL), and Goodpasture Syndrome, or a combination thereof. In certain embodiments, the ADR associated with the one or more checkpoint inhibitor is SJS/TEN.

The details of one or more embodiments of the present invention are set forth in the description below. Further areas of applicability will become apparent from the description provided herein. It should be understood that the description and specific examples are intended for purposes of illustration only and are not intended to limit the scope of the present disclosure.

### DETAILED DESCRIPTION

The following description of technology is merely exemplary in nature of the subject matter, manufacture and use of one or more present inventions, and is not intended to limit the scope, application, or uses of any specific present technology claimed in this application or in such other applications as may be filed claiming priority to this application, or patents issuing therefrom. The following definitions and non-limiting guidelines must be considered in reviewing the description of the technology set forth herein.

The term “antibody,” as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hyper variability, termed complementarity determining regions (CDRs), interspersed with regions that are more conserved, termed framework regions (FR). Each variable region (VH or VL) contains 3 CDRs, designated CDR1, CDR2 and CDR3. Each variable region also contains 4 framework sub-regions, designated FR1, FR2, FR3 and FR4. The term antibody includes all types of antibodies, including, for example, IgG and IgM. In some embodiments, the antibodies are IgM and in some embodiments, the IgM form polymerized pentamers.

As used herein, the term “antibody fragments” and “antigen-binding fragment.” in reference to an antibody, refers to a portion of an intact antibody that is able to bind the same antigen as the intact antibody. Examples of antibody fragments include, but are not limited to, linear antibodies, single-chain antibody molecules (scFv), Fv, Fab and F(ab′)2 fragments, and multispecific antibodies formed from antibody fragments. The antibody fragments preferably retain at least part of the heavy and/or light chain variable region.

The term “anti-αβ TCR antibody or antibody fragment” refers to an antibody or antibody fragment that binds the alpha chain of the human T-cell receptor, the beta chain of the human T-cell receptor, or both the alpha and beta chains of the human T-cell receptor.

As used herein, the phrase “TOL101 antibody” refers to a murine anti-αβ TCR monoclonal IgM antibody which binds to a human αβ TCR and is produced from the hybridoma TOL101 Master Cell Bank (MCB). As used herein, the term “master cell bank” refers to a culture of fully characterized cells processed together to ensure uniformity and stability. Typically, a MCB is a hybridoma cell line that is tested and determined to provide a stable and uniform source of a particular monoclonal antibody. TOL101 MCB was deposited on Nov. 2, 2012 at the ATCC and assigned ATCC accession number PTA-13293. As a IgM antibody, TOL-101 has a short half-life.

TOL101 has a Light chain encoded by the polynucleotide sequence of SEQ ID NO: 3:

ATGGATTTTCAAGTGCAGATTTTCAGCTTCCTGCTAATCAGTGCCTCAGT
CATAATATCCAGAGGACAAATTGTTCTCACCCAGTCTCCAGCAATCATGT
CTGCATCTCCAGGGGAGAAGGTCACCATGACCTGCAGTGCCAGCTCAAGT
GTAAGTTACATGCACTGGTACCAGCAGAAGTCAGGCACCTCCCCCAAAAG
ATGGATTTATGACACATCCAAACTGGCTTCTGGAGTCCCTGCTCGCTTCA
GTGGCAGTGGGTCTGGGACCTCTTACTCTCTCACAATCAGCAGCATGGAG
GCTGAAGATGCTGCCACTTATTACTGCCAGCAGTGGAGTAGTAACCCATT
CACGTTCGGCTCGGGGACAAAGTTGGAAATAAAACGGGCTGATGCTGCAC
CAACTGTATCCATCTTCCCACCATCCAGTGAGCAGTTAACATCTGGAGGT
GCCTCAGTCGTGTGCTTCTTGAACAACTTCTACCCCAAAGACATCAATGT
TAAGTGGAAGATTGATGGCAGTGAACGACAAAATGGCGTCCTGAACAGTT
GGACTGATCAGGACAGCAAAGACAGCACCTACAGCATGAGCAGCACCCTC
ACGTTGACCAAGGACGAGTATGAACGACATAACAGCTATACCTGTGAGGC
CACTCACAAGACATCAACTTCACCCATTGTCAAGAGCTTCAACAGGAATG
AGTGTTAG.

TOL101 has a Heavy chain encoded by the polynucleotide of SEQ ID NO:4:

ATGGAAAGGCACTGGATCTTTCTACTCCTGTTGTCAGTAACTGCAGGTGT
CCACTCCCAGGTTCAGCTGCAGCAGTCTCGGGCTGAACTGGCAAGACCTG
GGGCCTCAGTGAAGATGTCCTGCAAGGCTTCTGGCTACACCTTTACTAGC
TACACGATGCACTGGGTAAAACAGAGGCCTGGACAGGGTCTGGAATGGAT
TGGATACATTAATCCTAGCAGTGGTTATACTAATTACAATCAGAAGTTCA
AGGACAAGGCCACATTGACTGCAGACAAATCCTCCAGCACAGCCTACATG
CAACTGAGCAGCCTGACATCTGAGGACTCTGCAGTCTATTACTGTGCAAG
ATGGAGGGACGCGTACTATGCTATGGACTACTGGGGTCAAGGAACCTCAG
TCACCGTCTCCTCAGAGAGTCAGTCCTTCCCAAATGTCTTCCCCCTCGTC
TCCTGCGAGAGCCCCCTGTCTGATAAGAATCTGGTGGCCATGGGCTGCCT
GGCCCGGGACTTCCTGCCCAGCACCATTTCCTTCACCTGGAACTACCAGA
ACAACACTGAAGTCATCCAGGGTATCAGAACCTTTCCAACACTGAGGACA
GGGGGCAAGTACCTAGCCACCTCGCAGGTGTTGCTGTCTCCCAAGAGCAT
CCTTGAAGGTTCAGATGAATACCTGGTATGCAAAATCCACTACGGAGGCA
AAAACAGAGATCTGCATGTGCCCATTCCAGCTGTCGCAGAGATGAACCCC
AATGTAAATGTGTTCGTCCCACCACGGGATGGCTTCTCTGGCCCTGCACC
ACGCAAGTCTAAACTCATCTGCGAGGCCACGAACTTCACTCCAAAACCGA
TCACAGTATCCTGGCTAAAGGATGGGAAGCTCGTGGAATCTGGCTTCACC
ACAGATCCGGTGACCATCGAGAACAAAGGATCCACACCCCAAACCTACAA
GGTCATAAGCACACTTACCATCTCTGAAATCGACTGGCTGAACCTGAATG
TGTACACCTGCCGTGTGGATCACAGGGGTCTCACCTTCTTGAAGAACGTG
TCCTCCACATGTGCTGCCAGTCCCTCCACAGACATCCTAACCTTCACCAT
CCCCCCCTCCTTTGCCGACATCTTCCTCAGCAAGTCCGCTAACCTGACCT
GTCTGGTCTCAAACCTGGCAACCTATGAAACCCTGAATATCTCCTGGGCT
TCTCAAAGTGGTGAACCACTGGAAACCAAAATTAAAATCATGGAAAGCCA
TCCCAATGGCACCTTCAGTGCTAAGGGTGTGGCTAGTGTTTGTGTGGAAG
ACTGGAATAACAGGAAGGAATTTGTGTGTACTGTGACTCACAGGGATCTG
CCTTCACCACAGAAGAAATTCATCTCAAAACCCAATGAGGTGCACAAACA
TCCACCTGCTGTGTACCTGCTGCCACCAGCTCGTGAGCAACTGAACCTGA
GGGAGTCAGCCACAGTCACCTGCCTGGTGAAGGGCTTCTCTCCTGCAGAC
ATCAGTGTGCAGTGGCTTCAGAGAGGGCAACTCTTGCCCCAAGAGAAGTA
TGTGACCAGTGCCCCGATGCCAGAGCCTGGGGCCCCAGGCTTCTACTTTA
CCCACAGCATCCTGACTGTGACAGAGGAGGAATGGAACTCCGGAGAGACC
TATACCTGTGTTGTAGGCCACGAGGCCCTGCCACACCTGGTGACCGAGAG
GACCGTGGACAAGTCCACTGGTAAACCCACACTGTACAATGTCTCCCTGA
TCATGTCTGACACAGGCGGCACCTGCTATTGA.

TOL101 has a J chain encoded by the polynucleotide sequence of SEQ ID NO: 5:

ATGAAGACCCACCTGCTTCTCTGGGGAGTCCTGGCCATTTTTGTTAAGGC
TGTCCTTGTAACAGGTGACGACGAAGCGACCATTCTTGCTGACAACAAAT
GCATGTGTACCCGAGTTACCTCTAGGATCATCCCTTCCACCGAGGATCCT
AATGAGGACATTGTGGAGAGAAATATCCGAATTGTTGTCCCTTTGAACAA
CAGGGAGAATATCTCTGATCCCACCTCCCCACTGAGAAGGAACTTTGTAT
ACCATTTGTCAGACGTCTGTAAGAAATGCGATCCTGTGGAAGTGGAGCTG
GAAGATCAGGTTGTTACTGCCACCCAGAGCAACATCTGCAATGAAGACGA
TGGTGTTCCTGAGACCTGCTACATGTATGACAGAAACAAGTGCTATACCA
CTATGGTCCCACTTAGGTATCATGGTGAGACCAAAATGGTGCAAGCAGCC
TTGACCCCCGATTCTTGCTACCCTGACTAG.

The amino acid sequence of the TOL101 Light chain is according to SEQ ID NO: 6:

MDFQVQIFSFLLISASVHSRGQIVLTQSPAIMSASPGEKVTMTCSASSSV
SYMHWYQQKSGTSPKRWIYDTSKLASGVPARFSGSGSGTSYSLTISSMEA
EDAATYYCQQWSSNPFTFGSGTKLEIKRADAAPTVSIFPPSSEQLTSGGA
SVVCFLNNFYPKDINVKWKIDGSERQNGVLNSWTDQDSKDSTYSMSSTLT
LTKDEYERHNSYTCEATHKTSTSPIVKSFNRNEC

The amino acid sequence of the TOL101 Heavy chain is according to SEQ ID NO: 7:

MERHWIFLLLLSVTAGVHSQVQLQQSGAELARPGASVKMSCKASGYTFTS
YTMHWVKQRPGQGLEWIGYINPSSGYTNYNQKFKDKATLTADKSSSTAYM
QLSSLTSEDSAVYYCARWRDAYYAMDYWGQGTSVTVSSESQSFPNVFPLV
SCESPLSDKNLVAMGCLARDFLPSTISFTWNYQNNTEVIQGIRTFPTLRT
GGKYLATSQVLLSPKSILEGSDEYLVCKIHYGGKNRDLHVPIPAVAEMNP
NVNVFVPPRDGFSGPAPRKSKLICEATNFTPKPITVSWLKDGKLVESGFT
TDPVTIENKGSTPQTYKVISTLTISEIDWLNLNVYTCRVDHRGLTFLKNV
SSTCAASPSTDILTFTIPPSFADIFLSKSANLTCLVSNLATYETLNISWA
SQSGEPLETKIKIMESHPNGTFSAKGVASVCVEDWNNRKEFVCTVTHRDL
PSPQKKFISKPNEVHKHPPAVYLLPPAREQLNLRESATVTCLVKGFSPAD
ISVQWLQRGQLLPQEKYVTSAPMPEPGAPGFYFTHSILTVTEEEWNSGET
YTCVVGHEALPHLVTERTVDKSTGKPTLYNVSLIMSDTGGTCY

The amino acid sequence of the TOL101 J chain is according to SEQ ID NO: 8:

MKTHLLLWGVLAIFVKAVLVTGDDEATILADNKCMCTRVTSRIIPSTEDP
NEDIVERNIRVVPLNNRENISDPTSPLRRNFVYHLSDVCKKCDPVEVELE
DQVVTATQSNICNEDDGVPETCYMYDRNKCYTTMVPLRYHGETKMVQAAL
TPDSCYPD

As used herein a “αβ TCR” can include a heterodimer of a mammalian α-subunit and a mammalian β-subunit of a mammalian TCR. In some embodiments, the mammalian α-subunit can comprise the amino acid sequence of a human α-subunit, for example, an amino acid sequence of SEQ ID NO:1:

MAKTTQPNSMESNEEEPVHLPCNHSTISGTDYIHWYRQLPSQGPEYVIHG
LTSNVNNRMASLAIAEDRKSSTLILHRATLRDAAVYYCILPLAGGTSYGK
LTFGQGTILTVHPNIQNPDPAVYQLRDSKSSDKSVCLFTDFDSQTNVSQS
KDSDVYITDKTVLDMRSMDFKSNSAVAWSNKSDFACANAFNNSIIPEDTF
FPSPESS.

In some embodiments, the mammalian β-subunit can comprise the amino acid sequence of a human β-subunit, for example, an amino acid sequence of SEQ ID NO:2:

MAGSHMGVSQSPRYKVAKRGQDVALRCDPISGHVSLFWYQQALGQGPEFL
TYFQNEAQLDKSGLPSDRFFAERPEGSVSTLKIQRTQQEDSAVYLCASSL
GQAYEQYFGPGTRLTVTEDLKNVFPPEVAVFEPSEAEISHTQKATLVCLA
TGFYPDHVELSWWVNGKEVHSGVSTDPQPLKEQPALNDSRYCLSSRLRVS
ATFWQNPRNHFRCQVQFYGLSENDEWTQDRAKPVTQIVSAEAWGRADCTS
GD DDDK.

In some embodiments, an illustrative human αβ TCR can be a heterodimer comprising the subunits αβ of SEQ ID NOs: 1 & 2. In some embodiments, a human αβ TCR or fragment thereof comprises at least a portion of SEQ ID NOs: 1 & 2. In some embodiments, the human αβ TCR can include a portion or fragment that includes at least a portion of SEQ ID NO: 1 or 2.

As used herein, the terms “complementarity determining region” and “CDR” refer to the regions that are primarily responsible for antigen-binding. There are three CDRs in a light chain variable region (CDRL1, CDRL2, and CDRL3), and three CDRs in a heavy chain variable region (CDRH1, CDRH2, and CDRH3). The residues that make up these six CDRs have been characterized by Kabat and Chothia as follows: residues 24-34 (CDRL1), 50-56 (CDRL2) and 89-97 (CDRL3) in the light chain variable region and 31-35 (CDRH1), 50-65 (CDRH2) and 95-102 (CDRH3) in the heavy chain variable region; Kabat et al., (1991) Sequences of Proteins of Immunological Interest, 5th Ed. Public Health Service, National Institutes of Health, Bethesda. Md., herein incorporated by reference; and residues 26-32 (CDRL), 50-52 (CDRL2) and 91-96 (CDRL3) in the light chain variable region and 26-32 (CDRH1), 53-55 (CDRH2) and 96-101 (CDRH3) in the heavy chain variable region; Chothia and Lesk (1987) J. Mol. Biol. 196: 901-917, herein incorporated by reference. In certain embodiments, the terms “complementarity determining region” and “CDR” as used herein, include the residues that encompass both the Kabat and Chothia definitions (i.e., residues 24-34 (CDRL1), 50-56 (CDRL2), and 89-97 (CDRL3) in the light chain variable region; and 26-35 (CDRH1), 50-65 (CDRH2), and 95-102 (CDRH3)). Also, unless specified, as used herein, the numbering of CDR residues is according to Kabat. In certain embodiments, the present invention provides humanized antibodies composed of the six CDRs from TOL101, within a human framework (e.g., the deposited hybridomas are sequenced and humanized antibodies are assembled recombinantly according to techniques known in the art).

As used herein, the term “framework” refers to the residues of the variable region other than the CDR residues as defined herein. There are four separate framework sub-regions that make up the framework: FR1, FR2, FR3, and FR4. In order to indicate if the framework sub-region is in the light or heavy chain variable region, an “L” or “H” may be added to the sub-region abbreviation (e.g., “FRL1” indicates framework sub-region 1 of the light chain variable region). Unless specified, the numbering of framework residues is according to Kabat. It is noted that, in certain embodiments, the anti-αβ TCR antibodies or fragments thereof may have less than a complete framework (e.g. they may have a portion of a framework that only contains one or more of the four sub-regions).

As used herein, the term “fully human framework” means a framework with an amino acid sequence found naturally in humans. Examples of fully human frameworks, include, but are not limited to, KOL, NEWM, REI, EU, TUR, TEI, LAY and POM (See, e.g., Kabat et al., (1991) Sequences of Proteins of Immunological Interest, US Department of Health and Human Services, NIH, USA; and Wu et al., (1970) J. Exp. Med. 132, 211-250, both of which are herein incorporated by reference). In certain embodiments, the humanized antibodies of the present invention have fully human frameworks, or frameworks with one or more amino acids changed to accommodate the murine CDRs of TOL101.

As used herein, “Humanized” antibodies refer to a chimeric molecule, generally prepared using recombinant techniques, having an antigen binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the complementarity determining regions (CDRs) grafted onto appropriate framework regions in the variable domains. Antigen binding sites may be wild type or modified by one or more amino acid substitutions. This generally eliminates the constant region as an immunogen in human individuals, but the possibility of an immune response to the foreign variable region generally remains. Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the antigens in question and determine binding capability, flanked by four framework regions (FRs) which are relatively conserved in a given species and which putatively provide a scaffolding for the CDRs. When nonhuman antibodies are prepared with respect to a particular antigen, the variable regions can be “reshaped” or “humanized” by grafting CDRs derived from nonhuman antibody on the FRs present in the human antibody to be modified. Application of this approach to various antibodies has been reported by Sato, K., et al., (1993) Cancer Res 53:851-856. Riechmann, L., et al., (1988) Nature 332:323-327: Verhoeyen, M., et al., (1988) Science 239:1534-1536; Kettleborough, C. A., et al., (1991) Protein Engineering 4:773-3783: Maeda, H., et al., (1991) Human Antibodies Hybridoma 2:124-134; Gorman, S. D., et al., (1991) Proc Natl Acad Sci USA 88:4181-4185; Tempest, P. R., et al., (1991) Bio/Technology 9:266-271; Co, M. S., et al., (1991) Proc Natl Acad Sci USA 88:2869-2873; Carter, P., et al., (1992) Proc Natl Acad Sci USA 89:4285-4289; and Co, M. S. et al., (1992) J Immunol 148:1149-1154, all of which are herein incorporated by reference. In some embodiments, humanized antibodies preserve all CDR sequences (for example, a humanized mouse antibody which contains all six CDRs from the deposited TOL101 antibody). In other embodiments, humanized antibodies have one or more CDRs (one, two, three, four, five, six) which are altered with respect to the original antibody (e.g., original TOL101 antibody), which are also termed one or more CDRs “derived from” one or more CDRs from the original antibody.

As used herein, the terms “subject” and “patient” refer to any animal, such as a mammal. The term “mammal” as used herein refers to any mammal classified as a mammal, including humans, non-human primates, apes, pigs, cows, goats, sheep, horses, dogs, cats and those mammals employed in scientific research commonly known in the art, for example, mice, rats, hamsters, rabbits, guinea-pigs, and ferrets. In a preferred embodiment of the invention, the mammal is a human.

As used herein, the term “purified” or “to purify” refers to the removal of contaminants from a sample. For example, αβ TCR specific antibodies may be purified by removal of contaminating non-immunoglobulin proteins; they are also purified by the removal of immunoglobulins that do not bind to the same antigen. The removal of non-immunoglobulin proteins and/or the removal of immunoglobulins that do not bind the particular antigen results in an increase in the percentage of antigen specific immunoglobulins in the sample. In another example, recombinant antigen-specific polypeptides are expressed in bacterial, eukaryotic or mammalian host cells and the polypeptides are purified by the removal of host cell proteins; the percentage of recombinant antigen-specific polypeptides is thereby increased in the sample.

As used herein, the term “Fc region” refers to a C-terminal region of an immunoglobulin heavy chain. The “Fc region” may be a native sequence Fc region or a variant Fc region (e.g., with increased or decreased effector functions).

As used herein, an Fc region may possess “effector functions” that are responsible for activating or diminishing a biological activity (e.g., in a subject). Examples of effector functions include, but are not limited to: C1q binding; complement dependent cytotoxicity (CDC). Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g. Fc binding assays, ADCC assays, CDC assays, etc.).

As used herein, an “isolated” antibody or antibody fragment is one that has been identified and separated and/or recovered from a component of its natural environment. Contaminant components of its natural environment are materials that would interfere with diagnostic or therapeutic uses for the antibody or fragment thereof, and may include enzymes, hormones, and other proteinaceous or non-proteinaceous solutes. In certain embodiments, the isolated antibody is purified (1) to greater than 95% by weight of polypeptides as determined by the Lowry method, and preferably, more than 99% by weight, (2) to a degree sufficient to obtain at least 15 residues of N-terminal or internal amino acid sequence by use of a spinning cup sequenator, or (3) to homogeneity by SDS-page under reducing or non-reducing conditions using Coomassie blue, or silver stain. An isolated antibody includes the antibody in situ within recombinant cells since at least one component of the polypeptide's natural environment will not be present. Ordinarily, however, an isolated antibody will be prepared by a least one purification step.

As used herein, the term “treatment” refers to both therapeutic treatment and prophylactic or preventative measures. Those in need of treatment include those already with the disorder as well as those in which the disorder is to be prevented.

The phrase “under conditions such that the symptoms are reduced” refers to any degree of qualitative or quantitative reduction in detectable symptoms of any disease treatable by αβ TCR antibodies, including but not limited to, a detectable impact on the rate of recovery from disease (e.g., rate of weight gain), or the reduction of at least one of the symptoms normally associated with the particular disease (e.g., symptoms of graft rejection). In certain embodiments, the αβ TCR antibodies of the present invention are administered to a subject under conditions such that symptoms of graft rejection or GVHD are reduced (e.g., as compared to not treating with the αβ TCR antibodies).

The headings (such as “Background” and “Summary”) and sub-headings used herein are intended only for general organization of topics within the present technology, and are not intended to limit the disclosure of the present technology or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. Any discussion of the content of references cited in the present disclosure is intended merely to provide a general summary of assertions made by the authors of the references, and does not constitute an admission as to the accuracy of the content of such references. All references cited in the “Description” and “References” sections of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present technology, or embodiments thereof, may alternatively be described using more limiting terms such as “consisting of” or “consisting essentially of” the recited ingredients.

Unless otherwise defined, all technical and scientific terms used herein have the same meaning as commonly understood by one of ordinary skill in the art to which this present technology belongs. Although methods and materials similar or equivalent to those described herein can be used in the practice or testing of the present technology, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

### Isolated Antibodies and Antibody Fragments Directed to αβ TCR

The term “antibody” is used in the broadest sense and specifically covers single anti-αβ TCR antibodies (including agonist, antagonist, and neutralizing or blocking antibodies) and anti-αβ TCR antibody compositions with polyepitopic specificity. “Antibody” as used herein includes intact immunoglobulin or antibody molecules, polyclonal antibodies, multispecific antibodies (i.e., bispecific antibodies formed from at least two intact antibodies) and immunoglobulin fragments (such as scFv, Fab, F(ab′)2, or Fv), so long as they exhibit any of the desired agonistic or antagonistic or functional or clinical properties described herein.

Antibodies are typically proteins or polypeptides which exhibit binding specificity to a specific antigen. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains [Chothia et al., J. Mol. Biol., 186:651-663 (1985); Novotny and Haber, Proc. Natl. Acad. Sci. USA, 82:4592-4596 (1985)]. The light chains of antibodies from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes.

There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively.

“Antibody fragments” comprise a portion of an intact antibody, generally the antigen binding or variable region of the intact antibody. Examples of antibody fragments include Fab, Fab′, F(ab′)2, and Fv fragments, diabodies, and multispecific antibodies formed from antibody fragments.

The term “variable” is used herein to describe certain portions of the variable domains which differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies [see Kabat. E. A. et al., Sequences of Proteins of Immunological Interest. National Institutes of Health, Bethesda, Md. (1987)]. The constant domains are not involved directly in binding an antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen.

The monoclonal antibodies herein include chimeric, hybrid and recombinant antibodies produced by splicing a variable (including hypervariable) domain of an anti-αβ TCR antibody with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)2, and Fv), so long as they exhibit the desired biological activity or properties. See, e.g. U.S. Pat. No. 4,816,567 and Mage et al., in Monoclonal Antibody Production Techniques and Applications, pp. 79-97 (Marcel Dekker, Inc.: New York, 1987).

Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature, 256:495 (1975), or may be made by recombinant DNA methods such as described in U.S. Pat. No. 4,816,567. The “monoclonal antibodies” may also be isolated from phage libraries generated using the techniques described in McCafferty et al., Nature, 348:552-554 (1990), for example.

“Humanized” forms of non-human (e.g. murine) antibodies are specific chimeric immunoglobulins, immunoglobulin chains, or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin.

A “human antibody” is one which possesses an amino acid sequence which corresponds to that of an antibody produced by a human and/or has been made using any of the techniques for making human antibodies known in the art or as disclosed herein. This definition of a human antibody includes antibodies comprising at least one human heavy chain polypeptide or at least one human light chain polypeptide, for example an antibody comprising murine light chain and human heavy chain polypeptides. Human antibodies can be produced using various techniques known in the art. In one embodiment, the human antibody is selected from a phage library, where that phage library expresses human antibodies (Vaughan et al. Nature Biotechnology, 14:309-314 (1996): Sheets et al. PNAS, (USA) 95:6157-6162 (1998)); Hoogenboom and Winter, J. Mol. Biol., 227:381 (1991), Marks et al., J. Mol. Biol., 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545.807, 5,545,806; 5,569,825; 5,625,126; 5,633,425; 5,661,016, and in the following scientific publications: Marks et al., Bio/Technology, 10: 779-783 (1992); Lonberg et al., Nature, 368: 856-859 (1994): Morrison, Nature, 368:812-13 (1994); Fishwild et al., Nature Biotechnology, 14: 845-51 (1996); Neuberger, Nature Biotechnology, 14: 826 (1996): Lonberg and Huszar, Intern. Rev. Immunol., 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985): Boerner et al., J. Immunol., 147 (1):86-95 (1991); and U.S. Pat. No. 5,750,373.

The anti-αβ TCR antibody or antibody fragment variable regions and/or CDRs of the present invention, and variants thereof, may be employed with any type of suitable human constant regions (e.g., for chimeric antibodies) or human framework (e.g., for humanized antibodies). In certain embodiments, the constant regions are of the IgM class. Preferably, the CDRs are used with fully human frameworks or framework sub-regions. For example, the NCBI web site contains the sequences for certain human framework regions. Examples of human VH sequences include, but are not limited to, VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, VH3-43, VH3-48, VH3-49, VH3-53, VH3-64, VH3-66, VH3-7, VH3-72, VH3-73, VH3-74, VH3-9, VH4-28, VH4-31, VH4-34, VH4-39, VH4-4, VH4-59, VH4-61, VH5-51, VH6-1, and VH7-81, which are provided in Matsuda et al., J Exp. Med. 1998 Dec. 7; 188(11):2151-62, that includes the complete nucleotide sequence of the human immunoglobulin chain variable region locus, herein incorporated by reference. Examples of human VK sequences include, but are not limited to, A1, A10, A11, A14, A17, A18, A19, A2, A20, A23, A26, A27, A3, A30, A5, A7, B2, B3, L1, L10, L11, L12, L14, L15, L16, L18, L19, L2, L20, L22, L23, L24, L25, L4/18a, L5, L6, L8, L9, O1, O11, O12, O14, O18, O2, O4, and O8, which are provided in Kawasaki et al., Eur J Immunol 2001 April; 31(4):1017-28; Schable and Zachau, Biol Chem Hoppe Seyler 1993 November; 374(11):1001-22; and Brensing-Kuppers et al., Gene 1997 Jun. 3; 191(2):173-81, all of which are herein incorporated by reference. Examples of human VL sequences include, but are not limited to, V1-11, V1-13, V1-16, V1-17, V1-18, V1-19, V1-2, V1-20, V1-22, V1-3, V1-4, V1-5, V1-7, V1-9, V2-1, V2-11, V2-13, V2-14, V2-15, V2-17, V2-19, V2-6, V2-7, V2-8, V3-2, V3-3, V3-4, V4-1, V4-2, V4-3, V4-4, V4-6, V5-1, V5-2, V5-4, and V5-6, which are provided in Kawasaki et al., Genome Res 1997 March; 7(3):250-61, herein incorporated by reference. Fully human frameworks can be selected from any of these functional germline genes. Generally, these frameworks differ from each other by a limited number of amino acid changes. These frameworks may be used with the CDRs from TOL101 or variants thereof. Additional examples of human frameworks which may be used with the CDRs of the present invention include, but are not limited to, KOL, NEWM, REI, EU, TUR, TEI, LAY and POM (See, e.g., Kabat et al., 1987 Sequences of Proteins of Immunological Interest, US Department of Health and Human Services, NIH. USA, and Wu et al., 1970, J. Exp. Med. 132, 211-250, both of which are herein incorporated by reference).

The term “Fc region” is used to define the C-terminal region of an immunoglobulin heavy chain which may be generated by papain digestion of an intact antibody. The Fc region may be a native sequence Fc region or a variant Fc region. Although the boundaries of the Fc region of an immunoglobulin heavy chain might vary, the human IgG heavy chain Fc region is usually defined to stretch from an amino acid residue at about position Cys226, or from about position Pro230, to the carboxyl-terminus of the Fc region (using herein the numbering system according to Kabat et al., supra). The Fc region of an immunoglobulin generally comprises two constant domains, a CH2 domain and a CH3 domain, and optionally comprises a CH4 domain.

By “Fc region chain” herein is meant one of the two polypeptide chains of an Fc region. The “CH2 domain” of a human IgG Fc region (also referred to as “Cγ2” domain) usually extends from an amino acid residue at about position 231 to an amino acid residue at about position 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985). The CH2 domain herein may be a native sequence CH2 domain or variant CH2 domain.

The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protroberance” in one chain thereof and a corresponding introduced “cavity” in the other chain thereof; see U.S. Pat. No. 5,821,333). Such variant CH3 domains may be used to make multispecific (e.g. bispecific) antibodies as herein described.

“Hinge region” is generally defined as stretching from about Glu216, or about Cys226, to about Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions. The hinge region herein may be a native sequence hinge region or a variant hinge region. The two polypeptide chains of a variant hinge region generally retain at least one cysteine residue per polypeptide chain, so that the two polypeptide chains of the variant hinge region can form a disulfide bond between the two chains. The preferred hinge region herein is a native sequence human hinge region, e.g. a native sequence human IgG1 hinge region.

A “functional Fc region” possesses at least one “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95% sequence identity therewith.

“Antibody-dependent T-cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target T-cell and subsequently cause lysis of the target T-cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol., 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA), 95:652-656 (1998).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T-cells and neutrophils; with PBMCs and NK cells being preferred. The effector cells may be isolated from a native source thereof, e.g. from blood or PBMCs as described herein.

The terms “Fc receptor” and “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daeron, Annu. Rev. Immunol., 15:203-234 (1997)). FcRs are reviewed in Ravetch and Kinet, Annu. Rev. Immunol., 9:457-92 (1991); Capel et al., Immunomethods, 4:25-34 (1994); and de Haas et al., J. Lab. Clin. Med., 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol., 117:587 (1976); and Kim et al., J. Immunol., 24:249 (1994)).

“Complement dependent cytotoxicity” and “CDC” refer to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods, 202:163 (1996), may be performed.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology, 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene, 169:147-155 (1995), Yelton et al. J. Immunol., 155:1994-2004 (1995); Jackson et al., J. Immunol., 154(7):3310-9 (1995); and Hawkins et al, J. Mol. Biol., 226:889-896 (1992).

The term “immunospecific” as used in “immunospecific binding of antibodies” for example, refers to the antigen specific binding interaction that occurs between the antigen-combining site of an antibody and the specific antigen recognized by that antibody.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone: parathyroid hormone: thyroxine; insulin; proinsulin: relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin: placental lactogen; tumor necrosis factor-alpha and -beta: mullerian-inhibiting substance; mouse gonadotropin-associated peptide: inhibin; activin; vascular endothelial growth factor; integrin; thrombopoietin (TPO); nerve growth factors such as NGF-alpha; platelet-growth factor; transforming growth factors (TGFs) such as TGF-alpha and TGF-beta; insulin-like growth factor-I and -II: erythropoietin (EPO); osteo inductive factors; interferons such as interferon-alpha, -beta and -gamma colony stimulating factors (CSFs) such as macrophage-CSF (M-CSF): granulocyte-macrophage-CSF (GM-CSF); and granulocyte-CSF (G-CSF): interleukins (ILs) such as IL-1, IL-1alpha, IL-2, IL-3, IL-4, IL-5, IL-6, IL-7, IL-8, IL-9, IL-10, IL-11, IL-12; a tumor necrosis factor such as TNF-alpha or TNF-beta; and other polypeptide factors including LIF and kit ligand (KL). As used herein, the term cytokine includes proteins from natural sources or from recombinant T-cell culture and biologically active equivalents of the native sequence cytokines.

The terms “treating,” “treatment.” and “therapy” as used herein refer to curative therapy; therapy that reduces or ameliorates symptoms of a disease or condition: prophylactic therapy; and preventative therapy.

The term “therapeutically effective amount” refers to an amount of an active agent (e.g. an antibody or antibody fragment) or drug effective to treat a disease or disorder in a mammal. In the case of a T cell mediated cutaneous condition, the therapeutically effective amount of the active agent or drug may reduce the number of activated T cells, (for example αβ TCR+ T-cells); increase the number and/or activity of Treg-cells, reduce the production of inflammatory cytokines, or pro-immune cytokines, such as IL-2, interferon-γ, or TNF-α from activated T-cells; inhibit (i.e., slow to some extent and preferably stop) proliferation of activated T-cells; reduce the expression of CD3 on the surface of αβ TCR+ T-cells to below 50/mm3, preferably below 25/mm3; and/or relieve to some extent, one or more of the symptoms associated with the T cell mediated cutaneous condition. To the extent the antibody or antibody active agent or drug may prevent activation and expansion of αβ TCR+ T-cells, it may be anti-inflammatory and/or autoantigen tolerance inducing. For treatment of T cell mediated cutaneous conditions, efficacy in vivo can, for example, be measured by assessing the amount of cytokines such as IL-2, interferon-γ, or TNF-α from activated T-cells and the depletion of functional CD3 molecules on the surface of αβ TCR+ T-cells. In some instances, a “therapeutically effective amount” refers to an amount of an active agent (e.g. an anti αβ TCR antibody or antibody fragment) e.g. TOL101, or a secondary adjunct drug effective to abrogate, reduce, cease or prevent the severity of cutaneous conditions as measured by abrogation, reduction, cessation or prevention of cutaneous necrosis, blistering and/or detachment of skin.

The term “Human-anti-mouse antibody response” or “HAMA” refers to an immunologic response against a murine antibody following administration of a murine antibody to a human subject. Typically, mouse antibodies are recognized as foreign by the human immune system and thus they provoke the Human Anti-Mouse Antibody or HAMA response. The HAMA response interferes with the efficacy of the mouse antibody and can cause severe adverse symptoms in the recipient. The HAMA response may also interfere with the use of other murine-based therapeutics or diagnostics that may subsequently be administered to the patient. Methods for measuring HAMA and/or diagnosing HAMA in patients are well known in the art. See, e.g., ImmuSTRIP® HAMA IgG ELISA Test System (Catalog Number 10016; IMMUNOMEDICS®, INC. 300 American Road, Morris Plains, N.J. 07950) or HAMA (human anti-mouse antibodies) ELISA (IgG and IgM HAMA, Catalog Number 43-HAMHU-E01, ALPCO DIAGNOSTICS, 26-G Keewaydin Drive, Salem, N.H. 03079) or Gruber et. al, Cancer Res., 60: 1921-1926 (2000).

Exemplary Anti-αβ TCR Antibodies and their Properties

The invention disclosed herein has a number of exemplary embodiments. A variety of the typical embodiments of the invention are described below. The following embodiments are offered for illustrative purposes only, and are not intended to limit the scope of the present invention in any way. In certain embodiments of the methods, assays and compositions of the present invention, the anti-αβ TCR antibody or antibody fragment thereof comprises T10B9, MEDI-500 or TOL101 antibodies or fragments thereof. TOL101 is an isolated mouse IgM monoclonal antibody which binds to a αβ TCR and is produced by the hybridoma TOL101 MCB which is deposited with ATCC under the accession number PTA-13293.

In some embodiments of the present invention, the anti-αβ TCR antibody or antibody fragment thereof comprises an IgM backbone. Most therapeutic monoclonal antibodies are IgG antibodies which have a longer half life, e.g. about 14 to 21 days. IgM antibodies have a shorter half life, e.g. about 24 hours, and are preferred in the methods and compositions of the present invention. Due to their short half-life, IgM antibodies would be cleared rapidly after exerting therapeutic effect and would not suppress the immune system for a long period of time thereby minimizing the risk of opportunistic infections.

In various embodiments, anti-αβ TCR antibodies of the present invention bind specifically to a mammalian αβ TCR, for example, a human αβ TCR. In some embodiments, the anti-αβ TCR antibodies of the present invention bind to CD3+ T cells without a γδ TCR. Furthermore, anti-αβ TCR antibodies of the present invention do not bind to cells expressing markers such as CD14, CD16, B220 and CD19, further highlighting their specificity for the αβ T-cell. For example, homogenous, robust and reproducible anti-αβ TCR antibody binding of TOL101 has been observed in over 130 clinical patients and 20 healthy volunteers, as well as to common immortalized T-cell lines. Because anti-αβ TCR antibodies of the present invention bind to the entire population of αβ T-cells in a homogenous manner, it is believed that the anti-αβ TCR antibodies of the present invention bind a constant region of the αβ TCR.

Other anti-αβ TCR antibodies known in the art, such as T10B9. 1A-31 (T10B9) and MEDI-500, are immunoglobulin M kappa murine monoclonal antibodies (mAb) directed against the alpha-beta (αβ) heterodimer of the T-lymphocyte receptor complex. T10B9 is commercially available from BD Pharminigen™ (San Diego, Calif., USA) as Catalog Numbers 561674, 555548, 5555547, 561673. T10B9 has a relatively short duration of action, depleting T-cells for 10 to 14 days, unlike the protracted depletion seen with thymoglobulin and Campath-1H. Anti-αβ TCR antibodies such as T10B9 and MEDI-500 are nonmitogenic (i.e. do not induce cell proliferation) in soluble form at low concentrations; however, high concentrations of antibody in soluble form, or either low or high concentrations of plate-bound antibody (i.e., crosslinked antibody) may induce cell proliferation (Brown et al. Clinical Transplantation 10; 607-613 [1996]). T10B9 and MEDI-500 are sometimes used interchangeably in the literature, and have each previously been tested as therapeutic antibodies for indications such as treatment for allograft rejection and hematological malignancies. However, the clinical use of these antibodies was associated with adverse events and significant (e.g. about 30%) human-anti-mouse antibody (HAMA) responses (Waid et al. Transplantation 64; 274-281 [1997]). Brown et al., 1996, showed that T10B9/MEDI500 did not induce the expression of CD25, potentially suggesting that these clones do not induce Treg cells. The present invention contemplates using T10B9 and MEDI-500 for treating T cell mediated cutaneous conditions where the dosage of these antibodies is adjusted to prevent or lower the occurrence of adverse events and HAMA responses.

TOL-101 is non-mitogenic at high and low concentrations and in both soluble and plate-bound form. TOL101 exhibits minimal immune stimulation, minimal cytokine release, and robust T cell suppression with minimal adverse events and minimal HAMA responses. TOL101 reduces circulating CD3+, CD4+ and CD8+ T cells with no discernible impact on other blood cell populations. Unlike other T-cell directed antibodies, T-cell recovery is rapid following the cessation of TOL101 dosing. TOL101 has a very short half-life of about 23 hours. Under certain conditions, TOL101 may promote induction of Treg cells. Without wishing to be bound by theory, it is thought that the posttranslational modifications of TOL101, including, for example, the glycosylation and/or conformation of the antibody, are at least in part responsible for the superior clinical efficacy and safety of the TOL101 antibody. Unlike other T cell directed antibodies and drugs, TOL101 exhibits very little safety concerns upon administration.

In some embodiments, anti-αβ TCR antibodies used in the present invention do not deplete the numbers of circulating CD3+ T-cells significantly (for example, by an amount greater than 10% when compared to a vehicle control) when administered systemically at doses ranging from 0 to 42 mg/mL per day for 1 to 5 days. Instead, without wishing to be bound by theory, it is thought that the anti-αβ TCR antibodies used in the present invention downregulate the CD3 complex on αβ TCR+ T cells, including the αβ TCR itself, thus rendering the T cells unable to respond to antigen. As used herein, the terms “T cell depletion” and “T cell deletion” refer to the reduction of T cell numbers (e.g., circulating T cells in a subject). T cell depletion or deletion may be achieved by inducing cell death in a T cell. In one embodiment, the dose of an anti-αβ TCR antibody is considered to be effective when the count of CD3+ T cells is <25 CD3+ T cells/mm³. In one embodiment, the dose of an anti-αβ TCR antibody is considered to be effective when the count of CD3+ T cells is reduced 50%, 75% or 90% of baseline levels.

The invention also contemplates the use of chimeric, humanized, and other variant antibodies as well as antibody fragments prepared from T10B9, MEDI-500 or TOL101. A general scheme for preparing these variant antibodies or antibody fragments is described below and at other places in the application.

### Humanized Antibodies

Generally, a humanized antibody has one or more amino acid residues introduced into it from a non-human source. These non-human amino acid residues are often referred to as “import” residues, which are typically taken from an “import” variable domain. Humanization can be essentially performed following the method of Winter and co-workers [Jones et al., Nature, 321:522-525 (1986); Richmann et al., Nature, 332:323-327 (1988), Verhoeyen et al., Science, 239:1534-1536 (1988)], by substituting rodent CDRs or CDR sequences for the corresponding sequences of a human antibody.

Accordingly, such “humanized” antibodies are chimeric antibodies wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

It is important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

### Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (PCT application publication Nos. WO 91/00360 and WO 92/200373: EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

### Antibody Fragments

In certain embodiments, the anti-αβ TCR antibody of the present invention (including murine, human and humanized antibodies, and antibody variants) is an antibody fragment. Various techniques have been developed for the production of antibody fragments. Traditionally, these fragments were derived via proteolytic digestion of intact antibodies (see, e.g., Morimoto et al., J. Biochem. Biophys. Methods 24:107-117 (1992) and Brennan et al., Science 229:81 (1985)). However, these fragments can now be produced directly by recombinant host cells. For example, Fab′-SH fragments can be directly recovered from E. coli and chemically coupled to form F(ab′)2 fragments (Carter et al., Bio/Technology 10:163-167 (1992)). In one embodiment, single chain variable fragments (scFv) are produced from E. coli using techniques known in the art. In another embodiment, the F(ab′)2 is formed using the leucine zipper GCN4 to promote assembly of the F(ab′)2 molecule. According to another approach, Fv, Fab or F(ab′)2 fragments can be isolated directly from recombinant host cell culture. A variety of techniques for the production of antibody fragments will be apparent to the skilled practitioner. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields an F(ab′)2 fragment that has two antigen combining sites and is still capable of cross-linking antigen.

The Fab fragments produced in the antibody digestion also contain the constant domains of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells.

In some embodiments, antibodies or antibody fragments are isolated from antibody phage libraries generated using the techniques described in, for example, McCafferty et al., Nature, 348: 552554 (1990). Clackson et al., Nature, 352:624-628 (1991) and Marks et al., J. Mol. Biol., 222: 581-597 (1991) describe the isolation of murine and human antibodies, respectively, using phage libraries. Subsequent publications describe the production of high affinity (nM range) human antibodies by chain shuffling (Marks et al, BioTechnology, 10: 779-783 (1992)), as well as combinatorial infection and in vivo recombination as a strategy for constructing ver large phage libraries (e.g., Waterhouse et al., Nuc. Acids. Res., 21: 2265-2266 (1993)). Thus, these techniques, and similar techniques, are viable alternatives to traditional monoclonal antibody hybridoma techniques for isolation of monoclonal antibodies.

Also, the DNA may be modified, for example, by substituting the coding sequence for human heavy- and light-chain constant domains in place of the homologous murine sequences (e.g., U.S. Pat. No. 4,816,567, and Morrison, et al., Proc. Nat. Acad. Sci. USA, 81: 6851 (1984), both of which are hereby incorporated by reference), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

### Amino Acid Sequence Variants of Antibodies

Amino acid sequence variants of the anti-αβ TCR antibodies are prepared by introducing appropriate nucleotide changes into the anti-αβ TCR antibody DNA, or by peptide synthesis. Such variants include, for example, deletions from, and/or insertions into and/or substitutions of, residues within the amino acid sequences of the anti-αβ TCR antibodies of the examples herein. Any combination of deletion, insertion, and substitution is made to arrive at the final construct, provided that the final construct possesses the desired characteristics. The amino acid changes also may alter post-translational processes of the humanized or variant anti-αβ TCR antibody, such as changing the number or position of glycosylation sites.

A useful method for identification of certain residues or regions of the anti-αβ TCR antibody that are preferred locations for mutagenesis is called “alanine scanning mutagenesis,” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with DR4 antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed anti-αβ TCR antibody variants are screened for the desired activity.

Amino acid sequence insertions include amino- and/or carboxyl-terminal fusions ranging in length from one residue to polypeptides containing a hundred or more residues, as well as intra-sequence insertions of single or multiple amino acid residues. Examples of terminal insertions include an anti-αβ TCR antibody with an N-terminal methionyl residue or the antibody fused to an epitope tag. Other insertional variants of the anti-αβ TCR antibody molecule include the fusion to the N- or C-terminus of the anti-αβ TCR antibody of an enzyme or a polypeptide which increases the serum half-life of the antibody.

Another type of variant is an amino acid substitution variant. These variants have at least one amino acid residue in the anti-αβ TCR antibody molecule removed and a different residue inserted in its place. The sites of greatest interest for substitutional mutagenesis include the hypervariable regions, but FR alterations are also contemplated. Conservative substitutions are indicated below. If such substitutions result in a change in biological activity, then more substantial changes may be introduced and the products screened.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties.

The following eight groups each contain amino acids that are regarded conservative substitutions for one another: 1) Alanine (A) and Glycine (G): 2) Aspartic acid (D) and Glutamic acid (E); 3) Asparagine (N) and Glutamine (Q): 4) Arginine (R) and Lysine (K); 5) Isoleucine (I), Leucine (L), Methionine (M) and Valine (V): 6) Phenylalanine (F), Tyrosine (Y) and Tryptophan (W); 7) Serine (S) and Threonine (T); and 8) Cysteine (C) and Methionine (M) (see, e.g., Creighton, Proteins, W.H. Freeman and Co., New York (1984)).

In some embodiments, conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gin/asn; gly/asp; gly/ala or pro; his/asn or gin; ile/leu or val; leu/ile or val; lys/arg or gln or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (1), Histidine (H); 5) Isoleucine (I), Leucine (L), Methionine (M), Valine (V); and 6) Phenylalanine (F), Tyrosine (Y), Tryptophan (W); (see also, e.g., Creighton, Proteins, W.H. Freeman and Company (1984); Schultz and Schimer. Principles of Protein Structure, Springer-Verlag (1979)). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.”

Non-conservative substitutions will entail exchanging a member of one of these classes for another class. Any cysteine residue not involved in maintaining the proper conformation of the humanized or variant anti-αβ TCR antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or in addition, it may be beneficial to analyze a crystal structure of the antigen-antibody complex to identify contact points between the antibody and human DR4. Such contact residues and neighboring residues are candidates for substitution according to the techniques elaborated herein. Once such variants are generated, the panel of variants is subjected to screening as described herein and antibodies with superior properties in one or more relevant assays may be selected for further development.

### Glycosylation Variants of Antibodies

Antibodies are glycosylated at conserved positions in their constant regions (Jefferis and Lund, Chem. Immunol. 65:111-128 [1997); Wright and Morrison, TibTECH 15:26-32 [1997]). The oligosaccharide side chains of the immunoglobulins affect the protein's function (Boyd et al., Mol. Immunol. 32:1311-1318 [1996); Wittwe and Howard, Biochem. 29:4175-4180 [1990]), and the intramolecular interaction between portions of the glycoprotein which can affect the conformation and presented three-dimensional surface of the glycoprotein (Hefferis and Lund, supra: Wyss and Wagner, Current Opin. Biotech. 7:409-416 [1996]). Oligosaccharides may also serve to target a given glycoprotein to certain molecules based upon specific recognition structures. For example, it has been reported that in agalactosylated IgG, the oligosaccharide moiety ‘flips’ out of the inter-CH2 space and terminal N-acetylglucosamine residues become available to bind mannose binding protein (Malhotra et al., Nature Med. 1:237-243 (1995]). Removal by glycopeptidase of the oligosaccharides from CAMPATH-1H (a recombinant humanized murine monoclonal IgG1 antibody which recognizes the CDw52 antigen of human lymphocytes) produced in Chinese Hamster Ovary (CHO) cells resulted in a complete reduction in complement mediated lysis (CMCL) (Boyd et al., Mol. Immunol. 32:1311-1318 [1996]), while selective removal of sialic acid residues using neuraminidase resulted in no loss of DMCL. Glycosylation of antibodies has also been reported to affect antibody-dependent-cellular cytotoxicity (ADCC). In particular, CHO cells with tetracycline-regulated expression of .beta.(1,4)-N-acetylglucosaminyltransferase III (GnTIII), a glycosyltransferase catalyzing formation of bisecting GlcNAc, was reported to have improved ADCC activity (Umana et al., Mature Biotech. 17:176-180 [1999]).

Glycosylation variants of antibodies are variants in which the glycosylation pattern of an antibody is altered. By altering is meant deleting one or more carbohydrate moieties found in the antibody, adding one or more carbohydrate moieties to the antibody, changing the composition of glycosylation (glycosylation pattern), the extent of glycosylation, etc. Glycosylation variants may, for example, be prepared by removing, changing and/or adding one or more glycosylation sites in the nucleic acid sequence encoding the antibody, and expressing and translating the nucleic acid in a prokaryotic cell expression system.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-αβ TCR antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-αβ TCR antibody.

The glycosylation (including glycosylation pattern) of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, significant variations in the glycosylation pattern of the antibodies can be expected (see, e.g. Hse et al., J. Biol. Chem. 272:9062-9070 [1997]). Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example using endoglycosidase H (Endo H). In addition, the recombinant host cell can be genetically engineered, e.g. make defective in processing certain types of polysaccharides. These and similar techniques are well known in the art. The glycosylation structure of antibodies can be readily analyzed by conventional techniques of carbohydrate analysis, including lectin chromatography, NMR, Mass spectrometry, HPLC, GPC, monosaccharide compositional analysis, sequential enzymatic digestion, and HPAEC-PAD, which uses high pH anion exchange chromatography to, separate oligosaccharides based on charge. Methods for releasing oligosaccharides for analytical purposes are also known, and include, without limitation, enzymatic treatment (commonly performed using peptide-N-glycosidase F/endo-α-galactosidase), elimination using harsh alkaline environment to release mainly O-linked structures, and chemical methods using anhydrous hydrazine to release both N- and O-linked oligosaccharides. In some embodiments, the anti-αβ TCR antibodies of the present invention can be glycosylated. In some embodiments of the present invention, the anti-αβ TCR antibodies of the present invention can be unglycosylated. In some embodiments, the antibody or antigen binding fragments of the invention include antibodies or antibody fragments that bind to human αβ TCR that have or are engineered to have the same glycosylation pattern as the antibody produced from the hybridoma TOL101 MCB (TOL101). By “same glycosylation pattern” or “equivalent glycosylation pattern” it is intended that the antibodies of the invention have the same number and/or type of glycosylation sites as the antibody produced from the hybridoma TOL101 MCB (TOL101) such that the overall glycosylation signature or N- and O-linked oligosaccharide composition of the antibodies of the invention is similar to the glycosylation signature or N- and O-linked oligosaccharide composition of the antibody produced from the hybridoma TOL101 MCB (TOL101), as measured using the traditional techniques disclosed herein, resulting in the antibodies of the invention having at least one of the improved functional or clinical properties of TOL101 described herein. In addition, the antibody or antigen binding fragments of the invention may be glycosylated at equivalent or corresponding residues as the antibody produced by the hybridoma TOL101 MCB (TOL101). By “equivalent or corresponding residues” it is contemplated that the antibody or antigen binding fragments of the invention are glycosylated at residues that are within 35, within 30, within 25, within 20, within 15, within 10, or within 5 amino acid residues of the glycosylated residue in the antibody produced by the hybridoma TOL101 MCB (TOL101) when the two antibody sequences are aligned using publicly available computer software and the residue numbers of the glycosylated residues are identified according to Kabat such that the antibodies or antigen binding fragments of the invention have at least one of the improved functional or clinical properties of TOL101 over the prior art as described herein. Examples of suitable computer software include programs include the “Staden Package”, “DNA Star”, “MacVector”, GCG “Wisconsin Package” (Genetics Computer Group, Madison, Wis.) and “NCBI toolbox” (National Center for Biotechnology Information). As discussed herein, methods of identifying, comparing, altering and/or engineering the glycosylation of an antibody are also well known in the art.

### Treatment Methods Using Anti-αβ TCR Antibodies and Antibody Fragments Thereof

The present invention provides methods for treating diseases or disorders where negative modulation of αβ TCR+ T-cells is desired. For example, in certain embodiments, the invention provides methods for treating T cell mediated cutaneous conditions by administering anti-αβ TCR antibodies. T cell mediated cutaneous conditions include, but are not limited to, severe cutaneous adverse reactions (SCARs) such as acute generalized exanthematous pustulosis (AGEP), drug reaction with eosinophilia and systemic symptoms (DRESS), also known as drug-induced hypersensitivity syndrome or hypersensitivity syndrome (DIHS/HSS), Stevens-Johnson's syndrome (SJS), and toxic epidermal necrolysis (TEN) and other conditions such as epidermolysis bullosa and pemphigus vulgaris. In the methods of the present invention, the anti-αβ TCR antibodies or antibody fragments thereof, can be administered to a subject (e.g. human) in need thereof, alone or in combination with still other secondary adjunctive therapeutic agents or techniques.

In some embodiments, the invention provides methods for treating immune mediated adverse drug reactions comprising administering an anti-αβ TCR antibody or a fragment thereof. T cell mediated adverse drug reactions include, but are not limited to, SJS, TEN, DRESS/DIHS, AGEP, idiosyncratic liver injury (IDILI) such as hepatocellular IDILI and cholestatic IDILI, and drug-induced autoimmunity such as Drug-Induced Lupus-like Syndrome, Drug-Induced Cutaneous Lupus, Drug-Induced leukocytoclastic vasculitis, Drug-Induced renal vasculitis, and Drug-Induced pulmonary vasculitis. The anti-αβ TCR antibodies or antibody fragments thereof can be administered alone or in combination with other secondary adjunctive therapeutic agents or techniques.

In some embodiments, the invention provides methods for treating autoimmune diseases comprising administering an anti-αβ TCR antibody or a fragment thereof. The autoimmune diseases that may be treated according to the invention include, but are not limited to, pemphigus vulgaris, epidermolysis bullosa acquisita, and Goodpasture syndrome. Other forms of epidermolysis bullosa (EB) such as genetically inherited EB can also be treated using methods of the invention.

In various embodiments, methods for treating the above-mentioned conditions comprise administering a therapeutically effective dose of at least one antibody selected from the group consisting of T10B9, MEDI-500, TOL-101, an antibody fragment thereof and other variants such as humanized antibodies derived from T10B9, MEDI-500, TOL-101. In a particular embodiment, the antibody used in the methods of the invention is T10B9, MEDI-500, or TOL-101.

In certain embodiments of the invention, administration of anti-αβ TCR antibodies or antibody fragments thereof significantly down-regulates T cell activation, proliferation, and/or effects of activated T cells such as cytokine production and release cytotoxic mediators (e.g. perforin, granzyme, and granulysin).

Adverse drug reactions (ADRs) account for up to 10% of hospital admissions and are present with a diversity of phenotypes (Su and Chung, Curr. Rev. Immunol., 2014, 10, 33-40). Serious forms of cutaneous adverse reactions include SJS, TEN, DRESS (also known as DIHS) and AGEP. Other adverse drug reactions include liver injury, liver injury, hematological adverse reactions, and drug-induced autoimmunity (Uetrecht and Naisbitt, 2013). Although rare, these conditions remain a tough clinical problem due to significant morbidity and mortality.

In SJS and TEN, full thickness detachment of the epidermis may occur as a result of severe epidermal necrolysis. Clinical manifestations of SJS and TEN further include a rash featuring target-like lesions, and mucositis involving the ocular, oropharyngeal and genital surfaces. The patient is also systemically unwell with fever and malaise. Histological studies indicate that there is extensive apoptosis of keratinocytes that leads to epidermal necrolysis (Pareira et al., J. Am. Acad. Dermatol., 2007, 56(2), 181-200).

DRESS/DIHS is characterised by fever, rash, lymphadenopathy, eosinophilia and/or other leukocyte abnormalities, and internal organ involvement such as hepatitis. Clinical manifestations of AGEP include fever and the rapid appearance of disseminated sterile pustules 3-5 days after the commencement of treatment with an offending drug. It is accompanied by a marked neutrophilia. Histological studies in AGEP reveal intraepidermal, usually subcorneal, pustules and an accompanying neutrophilic and lymphocytic infiltrate.

In addition to skin reactions, idiosyncratic or immune-mediated adverse drug reactions also include single organ pathologies. The most common serious IDILI involves the death of hepatocytes: this is referred to as hepatocellular IDILI. The time to onset is usually 1-3 months: however, sometimes the delay between starting the drug and the onset of IDILI can be more than 1 year (Bjornsson, 2010). The second type of liver injury caused by drugs is called cholestatic liver injury which is characterized by a greater increase in alkaline phosphatase and bilirubin relative to alanine transaminase.

Clinical manifestations of other cutaneous conditions in which T cells play an important role also include blisters and/or skin lesions. For example, clinical manifestations of epidermolysis bolluosa (EB) include skin fragility, blisters and scars and clinical manifestations in pemphigus vulgaris (PV) include extensive flaccid blisters and mucocutaneous erosions.

T cells are also implicated in the pathogenesis of Goodpasture's Syndrome, also known as anti-glomerular basement membrane disease (anti-GBM-disease), where patients develop autoantibodies against the type IV collagen protein, α3(IV)NC1, expressed in the glomerular basement membrane, alveolar basement membrane, and basement membranes in the testis, inner ear, eye and the choroid plexus. Genetic studies have revealed a strong link between anti-GBM disease and HLA-DRB1*1501 and DRB1*1502 implicating the role of T cells (Hellmark and Segelmark, “Diagnosis and classification of Goodpastures disease (anti-GBM),” 2014, Journal of Autoimmunity. (48-49), 108-112).

Currently, there is no specific disease-modifying treatment regime suggested for various immune-mediated adverse drug reactions and other conditions (e.g. EB, PV, Goodpasture syndrome) discussed above beyond supportive care. Additionally, although T cells have been shown to play a significant role in immune mediated adverse drug reactions, diseases such as EB, PV, and Goodpasture syndrome, and skin cancers such as CTCL T cell specific therapies have not yet been extensively explored for these conditions. The present invention provides methods for treating these T cell mediated conditions by administering an anti-αβ TCR antibody or fragment thereof, wherein the anti-αβ TCR antibody or fragment thereof is non-mitogenic, non-stimulatory in nature. In certain embodiments, the anti-αβ TCR antibody or fragment thereof suppresses T cell activation by reducing the expression of signaling components. In some embodiments, administration of the anti-αβ TCR antibody or fragment thereof does not deplete T cells. In some embodiments, administration of the anti-αβ TCR antibody or fragment thereof reduces the number of CD3+ T cells to <25/mm³. In some embodiments, administration of the anti-αβ TCR antibody or fragment thereof reduces the number of CD3+ T cells 50%, 75% or 90%/o of baseline. In some other embodiments, administration of the anti-αβ TCR antibody or fragment thereof induces formation of Treg cells. In certain embodiments, administration of the anti-αβ TCR antibody or fragment thereof reduces circulating CD3+, CD4+ and CD8+ T cells with no or minimal effect on other blood cell populations. In some embodiments, there is a rapid recover) of T cells following the cessation of the anti-αβ TCR antibody or fragment thereof. In some embodiments, the anti-αβ TCR antibody is T10B9, MEDI-500, or TOL-101.

Embodiments of the present invention provide for methods for ameliorating clinical manifestations experienced by a subject suffering from an immune mediated adverse drug reaction. In accordance with these embodiments, administration of the anti-αβ TCR antibody or fragment thereof to a subject suffering from an immune mediated adverse drug reactions will reduce acute manifestations of the reaction, duration of the patient's hospital stay and/or overall disease morbidity and mortality.

Embodiments of the present invention provide for methods for ameliorating clinical manifestations experienced by a subject suffering from T cell mediated cutaneous conditions. In accordance with these embodiments, administration of the anti-αβ TCR antibody or fragment thereof to a subject suffering from a T cell mediated cutaneous condition provides resolution of rash, blisters and skin lesions, prevents or reduces the formation of rash, blisters and skin lesions, prevents or reduces apoptosis of keratinocytes, prevents or reduces epidermal necrolysis, prevents or reduces infiltration of effector T cells in the skin, prevents or reduces secretion of pro-inflammatory cytokines (e.g. TNF-α, IFN-γ, IL-6, IL-2 etc.), prevents or reduces release of cytotoxic mediators (e.g., perforin, granzyme and granulysin), decreases the duration of the patient's hospital stay and/or reduces the overall disease morbidity and mortality of the condition.

As described above, in some embodiments, administration of anti-αβ TCR antibodies or fragments thereof results in reduction or suppression of signaling components in αβ TCR+ T-cells, (for example, human CD4+T helper or memory cells or CD8+T effector cells), that may lead to reduced or complete cessation of stimulation, proliferation and/or production of other inflammatory or autoreactive cells including NK cells, macrophoages, neutrophils and proinflammatory cytokines such as Tumor Necrosis Factor-alpha (TNF-α); interferon-gamma (IFN-γ): or interleukins associated with STAT activation, for example, IL-2, IL-4, IL-5, IL-6, IL-9 and/or IL-13. In various treatment methods employed herein, administering a therapeutically effective amount of an anti-αβ TCR antibody or antibody fragment thereof results in a decreased expression or release of proinflammatory cytokines from said TCR+ T-cell as when compared to the activation of the TCR+ T-cell exposed to a cognate antigen in the context of MHC and in the absence of the anti-αβ TCR antibody or antibody fragment thereof. Furthermore, in some embodiments, the selective inhibition results in an αβ TCR+ T-cell that is not depleted, but merely loses the expression of CD3, (for example, at least 3 doses of anti-αβ TCR antibodies, or anti-αβ TCR antibody fragments, is sufficient to reduce the CD3+ count in the allograft transplant recipient to less than 25 cells per mm3), and continues to express CD2. Thus, administration of the anti-αβ TCR antibodies or fragments thereof according to the methods of the invention suppresses T cell activation, including stimulation, proliferation and/or production of other inflammatory or autoreactive cells and proinflammatory cytokines, without inducing T cell depletion, unlike other anti-T cell antibodies, such as OKT3, which deplete T-cells from circulation and may lead to severely immuno-compromised subjects.

In some embodiments, the present disclosure provides methods for treating or preventing immune-related adverse events (irAEs) associated with treatment of patients with immune-modulating drugs such as checkpoint inhibitors. Checkpoint inhibitors are drugs that block immune suppressive proteins on cancer cells and/or block the T cell proteins that bind such inhibitors, in order to circumvent immune suppression properties of cancer cells. Checkpoint inhibitors include antibodies or fragments thereof directed to PD-1. PD-L1 (B7-HI), and CTLA-4. Exemplary checkpoint inhibitors include ipilimumab, (YERVOY™), nivolumab (OPDIVO®), pembrolizumab (KEYTRUDAT™), and atezolizumab (TECENTRIG™). Checkpoint inhibitors present unique spectrum of side effects termed immune-related adverse events (irAEs). irAEs are unpredictable and can be mild to lethal (severity grade 1, 2, 3 or 4). They occur occur in up to 60% of patients treated with Grade 3/4 adverse events seen in 21% of patients, irAEs can hit any organ The most common irAEs include rash, colitis, hepatitis, endocrinopathies, and pneumonitis. irAE also include rheumatic disease like inflammatory arthritis and sicca syndrome irAE treatment includes interruption/termination of the checkpoint inhibitor and treatment with corticosteroids, irAEs are associated with clonal expansion of cross-reactive T cells, which can result in mild skin rash to lethal organ-specific toxicities. Pathophysiology of irAE is considered similar to that of autoimmune diseases, wherein activated lymphocytes target self-antigens. Treatment with checkpoint inhibitors can lead to T cell diversification, irAEs are specifically associated with a more diverse T cell repertoire and with an increase in the number of T cell clonotypes. Immune repertoire diversity following immune checkpoint inhibition can be both detrimental and beneficial to patients with cancer.

Combining checkpoint inhibitors increases the incidence of irAE. For example, combination of ipilimumab and nivolumab was evaluated in CheckMate 067 phase III trial. In that trial, the incidence of grade 3 or 4 toxicity with the combination was increased compared with either single agent (55 versus 16 and 27 percent, respectively, for nivolumab and ipilimumab). Treatment-related adverse events were more common with the combination (36 versus 8 and 15 percent, respectively). Severe acute nephritis has also been noted after combination treatment. In some embodiments, the present disclosure provides methods for inhibiting the incidence and/or severity of irAEs associated with combined checkpoint inhibitor therapy or with checkpoint inhibitor therapy combined with other therapies.

In some embodiments, the αβ-TCR antibodies provided herein provide an effective treatment for irAEs at least because they exhibit (a) a potent immunosuppressant that specifically attenuates T cell responses; (b) a fast-acting immunosuppressant to short circuit the response and cut it off; and (c) a short-acting immunosuppressant. Since patients dosed with checkpoint inhibitors have cancer, the short-acting and potent immunosuppression and ability to reset the T cell response provided by the antibodies and fragments provided herein are advantageous. In some embodiments, the advantageous properties of the antibodies and fragments thereof provided herein allow for re-administration of drug or for administration of alternative drugs/treatments.

### Administration

The antibody is preferably administered to the mammal in a carrier: preferably a pharmaceutically-acceptable carrier. Suitable carriers and their formulations are described in Remington's Pharmaceutical Sciences, 16th ed., 1980, Mack Publishing Co., edited by Oslo et al. Typically, an appropriate amount of a pharmaceutically-acceptable salt is used in the formulation to render the formulation isotonic. Examples of the carrier include saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of antibody being administered.

The anti-αβ TCR antibodies or antibody fragments thereof can be administered to the subject by injection (e.g., intravenous, intraperitoneal, subcutaneous, intramuscular, intraportal), or by other methods such as infusion that ensure its delivery to the bloodstream in an effective form. The antibody may also be administered by isolated perfusion techniques, such as isolated tissue perfusion, to exert local therapeutic effects. Local or intravenous injection is preferred.

Guidance in selecting appropriate doses for antibody is found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 0.01 mg/kg to up to 100 mg/kg of body weight or more per day, more preferably, from about 0.1 mg/kg to up to about 10 mg/kg, and even more preferably from about 0.2 mg/kg to about 0.7 mg/kg of body weight or more per day depending on the factors mentioned above.

In some embodiments, methods for treating T cell mediated cutaneous conditions can include administering an anti-αβ TCR antibody or antibody fragment thereof to a subject in need, in an amount from about 1 mg/day to about 200 mg/day, or from about 7 mg/day to about 58 mg/day or, from about 14 mg/day to about 45 mg/day, or from about 28 mg/day to about 42 mg/day, or an amount of 7 mg/day, 14 mg/day, 21 mg/day, 28 mg/day, 30 mg/day, 32 mg/day, 34 mg/day, 35 mg/day, 36 mg/day, 38 mg/day, 40 mg/day, 42 mg/day, 44 mg/day, 46 mg/day, 48 mg/day, 50 mg/day, 52 mg/day, 54 mg/day, 56 mg/day or 58 mg/day or more, or combinations thereof. As used herein, the integers 1 to 200 encompasses or includes, any integer or fraction thereof between the integers 1 and 200. For example, a daily dose of from about 7 mg/day to about 58 mg/day would naturally include all integers between this range, for example: 8, 10, 13, 27, 28, 29, 30, 45, 53 and 57 mg/day, and any fractional amounts thereof, for example, 3.5, 4.7, 5.25, 11.6, 22.1, 46.3 and 51.125 mg/day as merely examples of fractional amounts as contemplated between the integers 7 and 58. In some embodiments, a 3, 4, 5, 6, 7, 8, 9, 10, 11, 12, 13, or 14 day dosing schedule, can include an escalating and/or a diminishing dosing schedule which contemplates sequential daily doses that may be the same and/or may be different. In certain embodiments of the methods, assays and compositions of the present invention, the anti-αβ TCR antibody or antibody fragment thereof comprises T10B9, MEDI-500, TOL101 antibody or antibody fragment thereof which binds to a αβ TCR.

In some embodiments, methods for treating T cell mediated cutaneous conditions can include administering an anti-αβ TCR antibody or antibody fragment thereof to a subject in need, in an amount ranging from about 1 mg/day to about 200 mg/day, or from about 7 mg to about 58 mg per daily dose, or a fractional unit dose that added together comprises a daily dose, for example a daily dose of 28 mg can comprise two 14 mg doses administered at different times within a 24 hour period, for example twice a day, or every 12 hours. In some embodiments, the dosing regimen can comprise dosing a patient or subject in need thereof, with a composition, for example, a pharmaceutical composition, with a daily dose that essentially does not vary between day to day. In some embodiments, the subject is treated with a titrated daily dose that begins at day 0 with the highest daily dose, for example, 58 mg per day, or 56 mg/day or 42 mg/day and is titrated to the lowest daily dose, for example, 7 mg per day, or 14 mg/day over a period of three to five to six days. Exemplary dosing schedules are shown below in Tables 1-3.

TABLE 1
Exemplary 3 day diminishing dosing schedule

| | Daily | Daily | Daily | Daily | Daily | Daily | Daily | Daily | Daily | Daily |
|---|---|---|---|---|---|---|---|---|---|---|
| Day | Dose 1 | Dose 2 | Dose 3 | Dose 4 | Dose 5 | Dose 6 | Dose 7 | Dose 8 | Dose 9 | Dose 10 |
| 0 | 58 mg | 56 mg | 56 mg | 56 mg | 42 mg | 42 mg | 42 mg | 42 mg | 42 mg | 42 mg |
| 1 | 42 mg | 42 mg | 42 mg | 28 mg | 42 mg | 42 mg | 36 mg | 32 mg | 42 mg | 28 mg |
| 2 | 28 mg | 14 mg | 28 mg | 21 mg | 28 mg | 14 mg | 28 mg | 21 mg | 21 mg | 14 mg |

TABLE 2
Exemplary 6 day diminishing dosing schedule

| | Daily | Daily | Daily | Daily | Daily | Daily | Daily | Daily | Daily | Daily |
|---|---|---|---|---|---|---|---|---|---|---|
| Day | Dose 1 | Dose 2 | Dose 3 | Dose 4 | Dose 5 | Dose 6 | Dose 7 | Dose 8 | Dose 9 | Dose 10 |
| 0 | 58 mg | 56 mg | 56 mg | 42 mg | 42 mg | 42 mg | 42 mg | 42 mg | 42 mg | 42 mg |
| 1 | 42 mg | 42 mg | 42 mg | 28 mg | 42 mg | 42 mg | 36 mg | 32 mg | 42 mg | 28 mg |
| 2 | 28 mg | 42 mg | 28 mg | 21 mg | 42 mg | 42 mg | 28 mg | 21 mg | 21 mg | 21 mg |
| 3 | 21 mg | 28 mg | 21 mg | 14 mg | 28 mg | 28 mg | 21 mg | 21 mg | 21 mg | 14 mg |
| 4 | 14 mg | 21 mg | 14 mg | 14 mg | 14 mg | 14 mg | 21 mg | 14 mg | 14 mg | 7 mg |
| 5 | 14 mg | 14 mg | 7 mg | 14 mg | 14 mg | 7 mg | 14 mg | 14 mg | 7 mg | 7 mg |

TABLE 3
Exemplary 5 day diminishing dosing schedule

| | Daily | Daily | Daily | Daily | Daily | Daily | Daily | Daily | Daily | Daily |
|---|---|---|---|---|---|---|---|---|---|---|
| Day | Dose 1 | Dose 2 | Dose 3 | Dose 4 | Dose 5 | Dose 6 | Dose 7 | Dose 8 | Dose 9 | Dose 10 |
| 0 | 58 mg | 56 mg | 56 mg | 56 mg | 56 mg | 42 mg | 42 mg | 42 mg | 42 mg | 42 mg |
| 1 | 42 mg | 42 mg | 42 mg | 28 mg | 28 mg | 42 mg | 28 mg | 42 mg | 42 mg | 28 mg |
| 2 | 28 mg | 28 mg | 28 mg | 21 mg | 21 mg | 42 mg | 28 mg | 21 mg | 42 mg | 21 mg |
| 3 | 21 mg | 28 mg | 28 mg | 14 mg | 14 mg | 28 mg | 21 mg | 21 mg | 28 mg | 14 mg |
| 4 | 14 mg | 21 mg | 14 mg | 14 mg | 7 mg | 14 mg | 14 mg | 14 mg | 14 mg | 7 mg |

In some embodiments, the dosing regimen may comprise an escalation dosing schedule, wherein on day 0, the daily dose is the lowest daily dose to be administered to the subject. On the last day or someday within the interval of treatment, the daily dose is escalated to the highest daily dose. In one embodiment, the subject is administered an anti-αβ TCR antibody or antibody fragment thereof daily for a minimum of 3 days. In one embodiment, the subject is administered an anti-αβ TCR antibody or antibody fragment thereof daily for a minimum of 4 days. In one embodiment, the subject is administered an anti-αβ TCR antibody or antibody fragment thereof daily for a minimum of 5 days. In one embodiment, the subject is administered an anti-αβ TCR antibody or antibody fragment thereof daily for a minimum of 6 days. In one embodiment, the subject is administered an anti-αβ TCR antibody or antibody fragment thereof daily for a maximum of 10 days. In some embodiments, the subject is administered an anti-αβ TCR antibody or antibody fragment thereof until there is a significant resolution of the T cell mediated cutaneous condition. In one embodiment, the escalation dosing regimen can comprise the following dosing schedules exemplified in Tables 3-6:

TABLE 4
Exemplary- 3 day escalating dosing schedule

| | Daily | Daily | Daily | Daily | Daily | Daily | Daily | Daily | Daily | Daily |
|---|---|---|---|---|---|---|---|---|---|---|
| Day | Dose 1 | Dose 2 | Dose 3 | Dose 4 | Dose 5 | Dose 6 | Dose 7 | Dose 8 | Dose 9 | Dose 10 |
| 0 | 7 mg | 7 mg | 7 mg | 14 mg | 14 mg | 14 mg | 21 mg | 28 mg | 14 mg | 14 mg |
| 1 | 14 mg | 14 mg | 21 mg | 28 mg | 28 mg | 42 mg | 42 mg | 42 mg | 42 mg | 28 mg |
| 2 | 28 mg | 21 mg | 28 mg | 42 mg | 32 mg | 42 mg | 42 mg | 42 mg | 52 mg | 58 mg |

TABLE 5
Exemplary 6 day escalating dosing schedule

| | Daily | Daily | Daily | Daily | Daily | Daily | Daily | Daily | Daily | Daily |
|---|---|---|---|---|---|---|---|---|---|---|
| Day | Dose 1 | Dose 2 | Dose 3 | Dose 4 | Dose 5 | Dose 6 | Dose 7 | Dose 8 | Dose 9 | Dose 10 |
| 0 | 7 mg | 14 mg | 7 mg | 14 mg | 7 mg | 7 mg | 7 mg | 14 mg | 14 mg | 7 mg |
| 1 | 14 mg | 21 mg | 14 mg | 21 mg | 14 mg | 14 mg | 14 mg | 21 mg | 21 mg | 14 mg |
| 2 | 21 mg | 28 mg | 32 mg | 28 mg | 28 mg | 28 mg | 21 mg | 28 mg | 28 mg | 21 mg |
| 3 | 28 mg | 42 mg | 48 mg | 42 mg | 42 mg | 42 mg | 28 mg | 28 mg | 42 mg | 28 mg |
| 4 | 36 mg | 52 mg | 58 mg | 56 mg | 56 mg | 54 mg | 42 mg | 32 mg | 42 mg | 42 mg |
| 5 | 58 mg | 58 mg | 58 mg | 56 mg | 56 mg | 54 mg | 54 mg | 42 mg | 42 mg | 42 mg |

TABLE 6
Exemplary 5 day escalating dosing schedule

| | Daily | Daily | Daily | Daily | Daily | Daily | Daily | Daily | Daily | Daily |
|---|---|---|---|---|---|---|---|---|---|---|
| Day | Dose 1 | Dose 2 | Dose 3 | Dose 4 | Dose 5 | Dose 6 | Dose 7 | Dose 8 | Dose 9 | Dose 10 |
| 0 | 14 mg | 7 mg | 14 mg | 7 mg | 14 mg | 14 mg | 14 mg | 14 mg | 7 mg | 7 mg |
| 1 | 21 mg | 28 mg | 21 mg | 14 mg | 28 mg | 21 mg | 21 mg | 28 mg | 14 mg | 14 mg |
| 2 | 32 mg | 46 mg | 36 mg | 28 mg | 36 mg | 32 mg | 21 mg | 42 mg | 28 mg | 21 mg |
| 3 | 42 mg | 46 mg | 42 mg | 42 mg | 42 mg | 42 mg | 28 mg | 42 mg | 42 mg | 28 mg |
| 4 | 42 mg | 46 mg | 58 mg | 56 mg | 56 mg | 42 mg | 42 mg | 42 mg | 42 mg | 42 mg |

In some embodiments, the daily dose is the total daily dose, administered in one unit dose or multiple doses, for example, 2, 3 or 4 unit doses combined to arrive at the stated total daily dose.

In some embodiments, anti-αβ TCR antibodies or fragments thereof are administered within about 4, 5, 6, 7, 8, 9, or 10 days of disease onset. For example, anti-αβ TCR antibodies or fragments thereof are administered within about 4-10 days, about 5-10 days, about 6-10 days, about 7-10 days, or about 6-9 days of the onset of T cell mediated conditions. Onset of SJS, TEN, or SJS/TEN typically may occur up to about 3 weeks from the administration of the offending drug whereas onset of DRESS may occur from about 3 weeks to about 8 weeks from the administration of the offending drug. Anti-αβ TCR antibodies or fragments thereof are preferably administered within about the first 7 to 9 days of the disease onset. In certain embodiments, anti-αβ TCR antibodies or fragments thereof may also be administered after 10 days of disease onset or during the chronic phase of the disease. In certain embodiments, anti-αβ TCR antibodies or fragments thereof may also be administered within about 1, 2, 3, 4, 5, 6, 7, 8, 9, or 10 days of the patient's administration to the hospital.

In some embodiments, anti-αβ TCR antibodies or fragments thereof are administered subcutaneously. Administration of monoclonal antibodies usually involves large volumes of reconstituted formulations, such as 10, 20, 30, 40, 50 or more milliliters. The extracellular matrix of the subcutaneous tissue limits the injection of larger volumes (>1-2 mL). However, efforts have been focused on co-formulation of monoclonal antibodies (mAbs) with hyaluronidase for successful administration of mAbs subcutaneously (Jackish et al., Geburtshilfe Frauenheilkd, 2014, 74(4): 343-349, incorporated by reference herein in its entirety). Accordingly, the present invention provides subcutaneous formulation comprising an anti-αβ TCR antibody and hyaluronidase for treatment of T cell mediated cutaneous conditions. In some embodiments, anti-αβ TCR antibodies or fragments thereof may be administered subcutaneously in dosage amounts discussed above. In other embodiments, anti-αβ TCR antibodies or fragments thereof may be administered subcutaneously at a dose of about 5 to 20 mg/kg (e.g., 5, 6, 7, 8, 9, 10, 11, 12, 13, 14, 15, 16, 17, 18, 19, or 20 mg/kg) every 1, 2, 3, or 4 weeks. In some embodiments, anti-αβ TCR antibodies or fragments thereof are be administered subcutaneously once a week for the first three weeks followed by administration every other week.

In some embodiments, methods for treating immune mediated adverse drug reactions and T cell mediated cutaneous conditions can include administering at least three separate doses of anti-αβ TCR antibodies (e.g., IgG or IgM), or anti-αβ TCR antibody fragments thereof, to a subject, wherein the at least three separate doses are administered over three consecutive days, and wherein no two doses are administered on the same day. In other embodiments, the at least three separate doses comprises or consists of four separate doses, wherein the at least four separate doses are administered for four consecutive days, and wherein no two doses are administered on the same day. In particular embodiments, the at least three separate doses comprises or consists of five separate doses, wherein the at least five separate doses are administered for five consecutive days, and wherein no two doses are administered on the same day. In other embodiments, the at least three separate doses comprises or consists of six to fourteen separate doses, wherein the at least six to fourteen separate doses are administered for six to fourteen consecutive days, and wherein no two doses are administered on the same day.

In some embodiments, treating a subject with an immune mediated adverse drug reaction T cell mediated cutaneous condition can comprise administering at least a first dose of anti-αβ TCR antibodies (e.g., IgG or IgM), or anti-αβ TCR antibody fragments, to a subject, wherein the first dose is administered intravenously over at least 50 minutes or at least 70 minutes (e.g., 50-100 minutes: 70-200 minutes; 70-180 minutes; 70-140 minutes, or 70 . . . 140 . . . 180 . . . 200 minutes). In certain embodiments, the methods comprise administering at least a first dose of anti-αβ TCR antibodies (e.g., IgG or IgM), or anti-αβ TCR antibody fragments, to a subject, wherein the first dose is administered intravenously at a rate of between 0.05 mg/minute and 0.35 mg/minute (e.g., 0.05 . . . 0.1 . . . 0.2 . . . 0.3 . . . 0.35 mg/minute).

The anti-αβ TCR antibodies and antibody fragments may be administered by any suitable means, including parenteral, non-parenteral, subcutaneous, intradermal, intraocular, intravitreal, topical, intraperitoneal, intrapulmonary, and intranasal, and intralesional administration (e.g., for local treatment). Parenteral infusions include, but are not limited to, intramuscular, intravenous, subcutaneous, intradermal, intralesional, intraocular, intravitreal, intra-arterial, intraperitoneal, or subcutaneous administration. In addition, anti-αβ TCR antibodies and antibody fragments may be administered by pulse infusion, particularly with declining doses.

The anti-αβ TCR antibodies and antibody fragments can be incorporated into pharmaceutical compositions suitable for administration to a subject. For example, the pharmaceutical composition may comprise anti-αβ TCR antibodies and antibody fragments and a pharmaceutically acceptable carrier. As used herein, “pharmaceutically acceptable carrier” includes solvents, dispersion media, coatings, antibacterial and antifungal agents, isotonic and absorption delaying agents, and the like that are physiologically compatible. Examples of pharmaceutically acceptable carriers include one or more of the following: water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the anti-αβ TCR antibodies and antibody fragments.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. In one embodiment, anti-αβ TCR antibodies or fragments thereof are provided in lyophilized form. In another embodiment, anti-αβ TCR antibodies or fragments thereof are provided in a ready-to-use (RTU) solution form that is stable at room temperature or at refrigeration temperature, e.g., about 4° C., for an extended period of time, e.g., stable for about 1 month, about 3 months, about 6 months, about 12 months or more. In some embodiments, anti-αβ TCR antibodies or fragments thereof may be stabilized using pegylation. For example, the anti-αβ TCR antibodies or fragments thereof may be covalently attached to polyethylene glycol (PEG) polymer chains. Alternatively, compositions comprising the anti-αβ TCR antibodies or fragments thereof may comprise PEG as one of the excipients to provide stability to the antibodies/fragments.

Therapeutic compositions typically are sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody fragment) in the required amount in an appropriate solvent with one or a combination of ingredients enumerated above, as required, followed by sterile filtration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art (see, e.g., Sustained and Controlled Release Drug Delivery Systems, J. R. Robinson, ed., Marcel Dekker, Inc., New York, 1978).

The pharmaceutical compositions of the invention may include a “therapeutically effective amount” or a “prophylactically effective amount” of an antibody or antibody fragment of the invention. A “therapeutically effective amount” refers to an amount effective, at dosages and for periods of time necessary, to achieve the desired therapeutic result (e.g., prevent or reduce allograft rejection, treat, alleviate or prevent recurrence or occurrence of autoimmune and inflammatory symptoms and conditions). A therapeutically effective amount of the antibody or antibody fragment may vary according to factors such as the disease state, age, sex, and weight of the individual, and the ability of the antibody or antibody fragment to elicit a desired response in the individual. A therapeutically effective amount is also one in which any toxic or detrimental effects of the anti-αβ TCR antibodies or antibody fragment are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

In certain embodiments, the present invention provides compositions comprising an isolated antibody comprising an amino acid sequence of SEQ ID NOs: 6, 7, and 8, and wherein the isolated antibody binds to αβ TCR. In certain embodiments, the present invention provides compositions comprising an antibody which binds to a αβ TCR and competitively inhibits binding of the monoclonal antibody T10B9.1A-31 to the αβ TCR for the treatment of T cell mediated cutaneous conditions as described herein. In some embodiments, the present invention provides compositions comprising T10B9, MEDI-500 or TOL-101 formulated for intramuscular, intravenous, subcutaneous, intradermal, intralesional, intraocular, intravitreal, intra-arterial, intraperitoncal, or subcutaneous administration.

In certain embodiments, the present invention provides compositions comprising: isolated humanized monoclonal antibodies or fragments thereof comprising: i) at least one of the three complementary determining regions (CDRs) from the light chain variable region of the T10B9, MEDI-500 or TOL-101 antibody, ii) at least one of the three complementary determining regions (CDRs) from the heavy chain variable region of the T10B9, MEDI-500 or TOL-101 antibody, and iii) the constant regions from a human antibody. In further embodiments, the isolated humanized monoclonal antibodies or fragments thereof comprise: i) at least one, or at least two, or all three, of the three complementary determining regions (CDRs) from the light chain variable region of the T10B9, MEDI-500 or TOL-101 antibody, and ii) at least one, or at least two, or all three, of the three complementary determining regions (CDRs) from the heavy chain variable region of the T10B9, MEDI-500 or TOL-101 antibody.

In some embodiments, the present invention provides compositions comprising: isolated humanized monoclonal antibodies or fragments thereof comprising: i) the three complementary determining regions (CDRs) from the light chain variable region of the T10B9, MEDI-500 or TOL-101 antibody, ii) the three complementary determining regions (CDRs) from the heavy chain variable region of the T10B9, MEDI-500 or TOL-101 antibody, and iii) the constant regions from a human antibody. In further embodiments, the humanized monoclonal antibodies or fragments thereof, are lyophilized. In other embodiments, the compositions further comprise a physiologically tolerable buffer. In particular embodiments, the compositions further comprise or consist of at least one, two, or three of the following: i) sterile water; ii) L-arginine (e.g., about 100 mM L-arginine or 10-900 mM); iii) citrate (e.g., about 5 mM citrate, or about 1-25 mM citrate); iv) mannitol (e.g., about 4% mannitol (w/v) or about 1 to 30% w/v mannitol); and v) TWEEN or other non-ionic detergent (e.g., about 0.01% TWEEN 80, pH 7.0).

In further embodiments, T10B9, MEDI-500, TOL-101 or variants or fragments thereof, is present in the composition at between 14 mg and 52 mg, preferably between 28 mg and 52 mg, or present in the composition at 28 mg, 30 mg, 32 mg, 34 mg, 35 mg, 36 mg, 38 mg, 40 mg, 42 mg, 44 mg, 46 mg, 48 mg, or 50 mg.

### EXAMPLES

The following Examples are presented in order to provide certain exemplary embodiments of the present invention and are not intended to limit the scope thereof.

### Example 1: Clinical Trial of Patients Diagnosed with SJS, TEN, or SJS/TEN for Assessing Safety and Efficacy of TOL-101 Administration

Study design: Patients with a clinical diagnosis of SJS, TEN or SJS/TEN overlap will be enrolled for safety and efficacy studies.

For the safety phase of the study, safety of TOL-101 administration will be established using a small number (e.g. about 5-20) of patients. Patients may initially be administered with one-tenth and half the minimally anticipated biologic effect level (MABEL) of 2.8 mg or patients maybe initially administered escalating doses of TOL-101. The starting dose for the dose escalation will be 7 mg, 14 mg or 21 mg and will escalate to 42 mg as follows: dose escalating regimen 1 (7, 14, 21, 28, 42, 42, and 42 mg); dose escalating regimen 2 (14, 21, 28, 42, 42, and 42 mg); or dose-escalating regimen 3 (21, 28, 42, 42, and 42 mg).

Patients will receive one dose of TOL101 intravenously daily, or every other or every third day up to a maximum of about 5, 6, 7, 8, 9, or 10 doses. Maximum doses that a patient may receive may be extended based on efficacy response, CD3 counts and/or upon approval by Data Safety Monitoring Board (DSMB). TOL101 dosing will be fixed, i.e. the dose will not be based on the weight of the patient. The highest daily dose will not exceed 42 mg in the safety phase.

For the efficacy phase of the study, about 30-60 patients with a clinical diagnosis of SJS, TEN or SJS/TEN overlap will be administered with TOL-101 using a dosing regimen selected in the safety phase of the study.

An exemplary criteria for diagnosis of SJS and TEN is shown in Table 7 below:

TABLE 7
Diagnostic criteria for Stevens-Johnson syndrome (SJS)
Clinical entity
SJS is a severe mucocutaneous disorder characterized by erythema,
epidermal detachment (including blisters and erosions) and
enanthema accompanied by high fever. SJS is mainly caused by
a drug
Essential criteria (required)
Severe, hyperemic and/or hemorrhagic mucocutaneous lesions
Epidermal detachment involving less than 10% of the total body
surface area
High-grade fever (≥38.0° C.) in the absence of antipyretic therapy
Supportive findings
Flat atypical target lesions
Bilateral acute keratoconjunctivitis accompanied by ocular surface
epithelial defect and/or pseudomembranous formation
Histological evidence of epidermal necrosis
Diagnosis
Fulfillment of all three essential criteria is necessary for definite
diagnosis
Re-evaluation is necessary for final diagnosis due to the risk of
progression to its more extreme variant type, toxic epidermal
necrolysis (TEN)
Diagnostic criteria for toxic epidermal necrolysis (TEN)
Clinical entity
TEN is a severe mucocutaneous disorder characterized by extensive
erythema, epidermal detachment (including blisters and
erosions), and enanthema accompanied by high fever. The extent
of epidermal detachment is more than 10% of the total body
surface area. The cause of TEN is a drug in most patients
Essential criteria (required)
Epidermal detachment involving more than 10% of the total body
surface area
Exclusion of staphylococcal scalded skin syndrome
High-grade fever (≥38.0° C.) in the absence of antipyretic therapy
Supportive findings
Generalized macular or diffuse erythema
Enanthema including bilateral acute keratoconjunctivitis
accompanied by ocular surface epithelial defect and/or
pseudomembranous formation
Histological evidence of marked epidermal necrosis
Diagnosis
Fulfillment of all three essential criteria is necessary for definite
diagnosis

Administration of TOL-101: TOL101 will be provided in 14 mg lyophilized vials (Tolera Therapeutics, Inc., Kalamazoo, Mich., USA). TOL-101 will be reconstituted in sterile water for injection and diluted with normal saline.

TOL-101 will be administered by slow intravenous infusion. For example, 7 and 14 mg doses may be administered at a rate of about 0.1 mg/min, 21 or 28 mg doses may be administered at a rate of about 0.2 mg/min; and a 42 mg dose may be administered at a rate of about 0.3 mg/min. The infusion rate can also be determined as described by Getts et al., Clin Pharmacokinet, July 2014, 53(7):649-57, incorporated by reference herein in its entirety.

Clinical efficacy of TOL-101 dosing will be determined by the pharmacodynamic effect of TOL-101 on CD3+T lymphocyte counts. The dosing of TOL-101 will be considered efficacious if patients have CD3+ T cell numbers below 25 CD3+ counts per mm³ or CD3+ counts are reduced to 90% of baseline throughout the dosing interval. If not achieved, the dose of TOL-101 may be escalated.

TOL-101 will preferably be administered during the acute phase of the disease and/or immediately after administration to the hospital. For example, onset of SJS, TEN or SJS/TEN may occur within the first few days up to about three weeks from the administration of the offending drug and TOL-101 treatment will preferably be administered within about 7 to 9 days of the onset of SJS, TEN or SJS/TEN. It can also be administered within 7 to 10 days of a patient with SJS/TEN being admitted to the hospital. TOL-101 may be administered alone or in conjunction with antihistamines, pain medications, topical and/or systemic steroids and/or in conjunction with additional, non-approved therapies such as IVIG or cyclosporine.

End-point: End-points for efficacy measurements will include short and long-term assessments, e.g. morbidity to mortality, as we as the duration of the hospital stay.

For example, the primary efficacy end-point can be mortality compared to placebo and/or historical mortality rates as predicted by SCORTEN (SCORe of Toxic Epidermal Necrolysis), a validated prognostic score to predict outcomes in SJS/TEN patients. Mortality could be measured at 1 month, 2 month, 3 month, 6 month and 12 month post treatment with TOL101.

Secondary efficacy end-points may include progression of skin detachment, duration of hospitalization, severity of the disease, evaluated with the simplified acute physiology score [SAPS, a prognosis score calculated from seven clinical variables (age, heart rate, systolic blood pressure, body temperature, respiratory rate, urinary output per 24 h, Glasgow coma score) and seven biological variables (BUN, Hct, packed cell volume, WBC and plasma concentrations of glucose potassium, sodium, and bicarbonate)].

Alternatively, a severity of-illness scale that scores ophthalmic lesions, lip/oral lesions, cutaneous lesions and general condition, with a total score ranging from 0 to 39 as described by Aihara et al. (J Dermatol, 2015 August, 42(8):768-77) may be used. This rating system is shown in Table 8. Patients with a reduction of 6 or more from day 1 of the therapy will be considered responders.

Another alternative rating scale called an auxiliary score was defined by Sakula et al. (J Burn Care Res, 2011, 32: 237-245) to determine expected mortality in TEN patients. Criteria for this method are shown in Table 9.

TABLE 8
Severity-of-illness score described by Aihara et al.

| | Score 0 | Score 1 | Score 2 | Score 3 | Score 4 | Score 5 | Score 6 |
|---|---|---|---|---|---|---|---|
| Ophthalmic lesions | | | | | | | |
| Pseudomembrane | None | Slight pseudomembrane | Pseudomembrane is formed | Difficulty in opening the | — | — | — |
| formation | | formation | but the patient is able to | eyelids | | | |
| | | | open the eyelids | | | | |
| Conjunctival hyperemia | None | Mild conjunctival | Moderate conjunctival | Severe conjunctival | — | — | — |
| | | vascular hyperemia | vascular hyperemia | vascular hyperemia | | | |
| Lip/oral lesions | | | | | | | |
| Blood crust or | None | Erosion without blood | Erosion with blood crust or | Erosion with extensive | — | — | — |
| hemorrhage or | | crust or hemorrhage | hemorrhage on the lip | blood crust or hemorrhage | | | |
| oral erosion | | | | on the lip and in oral cavity | | | |
| Cutaneous lesions | | | | | | | |
| Effusion in the | Stopped/ | Slight | Mild | Severe | — | — | — |
| erosion/ulcer area | none | | | | | | |
| Hemorrhage in the | Stopped/ | Mild | Moderate | Severe | — | — | — |
| erosion/ulcer area | none | | | | | | |
| Extent of epidermal | 0% | <5% | ≥5%, <10% | ≥10%, <15% | ≥15%, | ≥20%, | ≥30% |
| detachment^{†} | | | | | <20% | <30% | |
| Extent of erythema^{‡} | 0% | <10% | ≥10%, <20% | ≥20%, <30% | ≥30%, | ≥40%, | ≥50% |
| | | | | | <40% | <50% | |
| Cutaneous/mucosal | None | Slight pain | Considerable pain | Intolerable pain, requiring | — | — | — |
| pain | | | | sedation | | | |
| General condition | | | | | | | |
| Oral intake | Normal | The patient eats more | The patient eats less than | The patient does not eat | — | — | — |
| | | than half | half. | meals (including nil | | | |
| | | | | p.o. status) | | | |
| Malaise | None | Mild | Moderate | Severe | — | — | — |
| Fever | <37.0° C. | ≥37.0° C., <37.5° C. | ≥37.5° C., <38.5° C. | ≥38.5° C. | — | — | — |

^{†}The extent of epidermal detachment is measured on the assumption that the patient's palm (including fingers) was equivalent to 1% of the body surface area. The epithelialized areas are excluded.
^{‡}The extent of erythema is measured on the assumption that the patient's palm (including fingers) was equivalent to 1% of the body surface area. The pigmented areas are excluded.

TABLE 9

| Variable | Weight |
|---|---|
| Age 31-55 years | 1 |
| Age 56-75 years | 2 |
| Age >75 years | 3 |
| Detached or compromised body surface | 1 |
| area >30% | |
| Presence of cancer or malignancy | 1 |
| Range of score | 0-5 |

The secondary end-points will be the changes over time in severity-of-illness score (e.g. 4, 7, 10, and 20 days), extent of epidermal detachment and extent of erythema until day 20. Sequelae will be monitored until discharge of patients.

Additionally, the following end points may be monitored:

Avulsed skin area (20 days) (secondary outcome, safety)

Erythematous area (20 days) (secondary outcome, safety)

30 day mortality

Safety labs and adverse events

Time-to-cutaneous re-epithelialization (up to 14 days)

Time-to-mucosal re-epithelialization (up to 14 days)

Time-to-cessation of epidermal necrosis (up to 14 days)

Safety (numeric cellulitis score)

Time to cessation of skin detachment (up to 14 days)

Time to 90% re-epithelialization (up to 14 days)

Percent Affected Surface Area & Percent Affected Surface Area Detached Skin (up to 14 days)

Additionally, the following endpoints may be monitored—Peripheral CD3+ T cell kinetics, serum cytokine levels (e.g., TNF, IL-6, IL-6) and skin biopsy (e.g., keratinocyte apoptosis, lymphocyte infiltration)

Patients will be monitored throughout the study for any serious adverse events and adverse events.

### Example 2: Treatment of Patients Diagnosed with Epidermolysis Bullosa (EB) Using TOL-101

Study design and administration of TOL-101: Patients with proven diagnosis of EB will be enrolled for safety and efficacy studies with escalating doses of TOL-101. TOL-101 may be administered alone or in conjunction with systemic or topical steroids. For every patient, a specific skin lesion or one or more pair of lesions will be selected to monitor the efficacy of the treatment.

Dosing amounts and dosing schedule of TOL-101 will be as described in Example 1. CD3+ counts<25/mm³ or CD3+ counts are reduced to 90% of baseline will be used as a marker for effective dosing of TOL-101.

End-point: The primary efficacy endpoint will be the higher degree of epithelialization between the treated group versus the control group as assessed by two independent blinded experts using digital macro-photographs of the selected skin lesions taken at specific intervals, e.g. day 0 (prior to the treatment), day 1 (the first day of treatment), day 4, day 7, day 14, day 21, and day 28. The blinded experts will evaluate re-epithelialization of the photographed lesions for each time point.

Secondary efficacy variables will be percentage of wound epithelialization as measured and documented at each time point. The degree of re-epithelialization will be calculated for each lesion as a ratio of re-epithelialized area to initial open lesion area. Additionally, touch sensitivity, itching, and exudation may be measured.

### Example 3: In Vitro Testing of TOL101 for SJS/TEN

Cells are isolated from SJS/TEN patients and tested to determine the effect of TOL101 on killing activity, proliferation, granulysin and/or cytokine release.

To test the effect of TOL101 on T cell granulysin release, PBMC are isolated from SJS/TEN patients in the recovery stage and cultured in 96-well U-bottom plates (1.0×10 cells per well) containing medium, culprit drugs or tolerant drugs in the presence or absence of TOL101. After 7 days of culture, the released granulysin levels in culture supernatant are measured by ELISA assay. The effect of TOL101 is also be tested by direct incubation with SJS blister cells in which granulysin is highly expressed by CTLs/NK cells. After 3 days co-culture of SJS blister cells with or without TOL101 (with or without culprit drug), the released granulysin levels in culture supernatant are measured by ELISA assay.

To assess the effect of TOL101 on cytoxicity, drug-specific CD8+CTLs were isolated from PBMCs or blister fluids obtained from patients with CBZ or allopurinol-induced SJS/TEN. The HLA-B*1502 or HLA-B*5801 expressing B cells are labeled with CFSE dyes and then the labeled B cells are incubated with drug-specific CTLs, CBZ or allopurinol in the presence or absence of TL101 with an effector cells/target cell ratio of 1:1 to 25:1. Samples are measured by flow cytometry and the specific lysis of target B cells is calculated.

For proliferation assays, PBMCs are isolated from SJS/TEN patients after recovery from the acute disease via Ficoll-Hypaque density gradient centrifugation. Mononuclear cells are seeded at 10⁶ cells/ml in triplicates in round bottom 96 well plates in a final volume of 200 μl in RPMI medium in the presence of 5% autologous serum and different drug concentrations. The cultures are incubated for 5 days at 37° C. in 5% CO2 and 1 uCi 3H thimidine is added to each well 18 h prior to cell harvesting. The cultures are harvested onto glass fiber filters and 3H incorporation is estimated by scintillation counting. Phytohemaglutinin and Tetanus Toxoid are used as positive controls. The stimulation index (SI) is calculated as the ratio between the mean values of counts per minute in cultures with antigen an those obtained without antigen A SI>2 is regarded as positive response to most drugs except betalactams (SI>3) and iodinated contrasts (SI>4) (Pichler and Tilch, Allergy 2004). To test the effect of TOL101, cultures will be established in parallel in the presence of 9 μg/ml (minimal effective concentration) and 50 g/ml of the antibody TOL101 or control mouse IgM.

For the degranulation assay, B cell lines from patients with well defined culprit drug (positive LTT using routine protocol) will be immortalized, and these LCL-B cells will be incubated with drug-specific CTLs (in vitro expanded polyclonal populations grown in the presence of the culprit drug for at least two weeks) or blister fluid cells, and the culprit drug, in the presence or absence of TOL101 with an effector cells/target cells ratio of 1:1. The cultures will be set in the presence of CD107-PE. Monensin will be added after 1 hour incubation. Upon a total 4 h culture, degranulation will be measured by flow cytometry analysis of CD107-PE in CD8+ T cells (expanded CTLs) and in CD8+ T cells and CD3-CD56+NK cells. Heat-inactivated FCS may be used. If CD3 is internalized with the TCR, fluorochrome conjugated-CD3 can be added together with CD107 at the beginning of the assay. The release of granulysin could be also evaluated by ELISA in the same cultures.

### REFERENCES

1. Uptodate: Toxicities associated with checkpoint inhibitor immunotherapy (http://www.uptodate.com/contents/toxicities-associated-with-checkpoint-inhibitor-immunotherapy?source=search_result&search=checkpoint+ inhibitors&selectedTitle=1%7E59)
2. Larkin, James, et al. “Combined nivolumab and ipilimumab or monotherapy in untreated melanoma.” N Engl J Med 2015.373 (2015): 23-34.
3. Murakami, Naoka, et al. “Severe acute interstitial nephritis after combination immune-checkpoint inhibitor therapy for metastatic melanoma.” Clinical kidney journal 9.3 (2016): 411-417.
4. Cha, Edward, et al. “Improved survival with T cell clonotype stability after anti-CTLA-4 treatment in cancer patients.” Science translational medicine 6.238 (2014): 238ra70-238ra70.
5. Oh, David Y., et al. “T cell repertoire diversification is associated with immune related toxicities following immune checkpoint inhibition in metastatic cancer patients.” Cancer Research 76.14 Supplement (2016): 4362-4362.
6. Friedman, Claire F., Tracy A. Proverbs-Singh, and Michael A. Postow. “Treatment of the Immune-Related Adverse Effects of Immune Checkpoint Inhibitors: A Review.” JAMA oncology (2016).
7. Calabrese. Leonard, and Vamsidhar Velcheti. “Checkpoint immunotherapy: good for cancer therapy, bad for rheumatic diseases.” (2016): annrheumdis-2016.
8. Cappelli, Laura C., et al. “Inflammatory arthritis and sicca syndrome induced by nivolumab and ipilimumab.” Annals of the Rheumatic Diseases (2016): annrheumdis-2016.
9. Villadolid, Jeryl, and Asim Amin. “Immune checkpoint inhibitors in clinical practice: update on management of immune-related toxicities.” Translational lung cancer research 4.5 (2015): 560.
10. Chen, T. W., et al. “A systematic review of immune-related adverse event reporting in clinical trials of immune checkpoint inhibitors.” Annals of Oncology 26.9 (2015): 1824-1829.
11. Chung, Wen-Hung, et al. “Granulysin is a key mediator for disseminated keratinocyte death in Stevens-Johnson syndrome and toxic epidermal necrolysis.” Nature medicine 14.12 (2008): 1343-1350.
12. Wei, Chun-Yu, et al. “Direct interaction between HLA-B and carbamazepine activates T cells in patients with Stevens-Johnson syndrome.” Journal of Allergy and Clinical Immunology 129.6 (2012): 1562-1569.
13. Ko, Tai-Ming, et al. “Shared and restricted T-cell receptor use is crucial for carbamazepine-induced Stevens-Johnson syndrome.” Journal of Allergy and Clinical Immunology 128.6 (2011): 1266-1276.

What is claimed is:

1. A method for treating an immune mediated adverse drug reaction (IM-ADR) in a subject in need thereof, comprising administering to the subject an anti-αβ T cell receptor (TCR) antibody or an antigen binding fragment thereof.

2. The method of claim 1 wherein the immune mediated adverse drug reaction is a T cell mediated hypersensitivity condition, a severe cutaneous adverse reactions (SCARs), an acute generalized exanthematous pustulosis (AGEP), drug reaction with eosinophilia and systemic symptoms (DRESS), Stevens-Johnson's syndrome (SJS), and toxic epidermal necrolysis (TEN), an idiosyncratic adverse drug reaction, or an idiosyncratic liver injury (IDILI).

3.-6. (canceled)

7. The method of claim 1, wherein the anti-αβ TCR antibody or antigen binding fragment thereof is an IgM antibody or an antigen binding fragment of the IgM antibody, or is non-mitogenic and non-stimulating in nature.

8. (canceled)

9. The method of claim 1, wherein the anti-αβ TCR antibody or antigen binding fragment is selected from the group consisting of T10B9, MEDI-500, and TOL-101.

10. The method of claim 9, wherein the anti-αβ TCR antibody or antigen binding fragment thereof is administered in a therapeutically effective amount, or is administered once per day, or is administered via intravenous, subcutaneous, intradermal, intralesional, intraocular, and/or intravitreal route.

11. (canceled)

12. (canceled)

13. The method of claim 1, wherein the anti-αβ TCR antibody or antigen binding fragment thereof is administered in an amount from about 7 mg/day to about 58 mg/day.

14. The method of claim 1 wherein the anti-αβ TCR antibody or antigen binding fragment thereof is administered in an amount of 7 mg/day, 14 mg/day, 21 mg/day, 28 mg/day, 30 mg/day, 32 mg/day, 34 mg/day, 35 mg/day, 36 mg/day, 38 mg/day, 40 mg/day, 42 mg/day, 44 mg/day, 46 mg/day, 48 mg/day, 50 mg/day, 52 mg/day, 54 mg/day, 56 mg/day or 58 mg/day, or combinations thereof.

15.-19. (canceled)

20. The method of claim 1, wherein the anti-αβ TCR antibody or antigen binding fragment thereof is administered according to a dosing schedule comprising: 14 mg at day 1, 21 mg at day 2, 28 mg at day 3, 42 mg at day 4, 42 mg at day 5, and 42 mg at day 6.

21. A method for treating a T cell mediated cutaneous condition in a subject in need thereof, comprising administering to the subject an anti-αβ T cell receptor (TCR) antibody or antigen binding fragment thereof.

22. The method of claim 21 wherein the T cell mediated cutaneous condition is epidermolysis bullosa (EB) or pemphigus vulgaris, or cutaneous T cell lymphoma (CTCL).

23. (canceled)

24. The method of claim 21, wherein the anti-αβ TCR antibody or antigen binding fragment thereof is selected from the group consisting of T10B9, MEDI-500, and TOL101.

25. The method of claim 21, wherein the anti-αβ TCR antibody or antigen binding fragment thereof is administered in an amount of 7 mg/day, 14 mg/day, 21 mg/day, 28 mg/day, 30 mg/day, 32 mg/day, 34 mg/day, 35 mg/day, 36 mg/day, 38 mg/day, 40 mg/day, 42 mg/day, 44 mg/day, 46 mg/day, 48 mg/day, 50 mg/day, 52 mg/day, 54 mg/day, 56 mg/day or 58 mg/day, or combinations thereof.

26. The method of claim 21, wherein the anti-αβ TCR antibody or antigen binding fragment thereof is administered according to a dosing schedule comprising: 14 mg at day 1, 21 mg at day 2, 28 mg at day 3, 42 mg at day 4, 42 mg at day 5, and 42 mg at day 6.

27. A method for treating an autoimmune disease in a subject in need thereof, comprising administering to the subject an anti-αβ T cell receptor (TCR) antibody or antigen binding fragment thereof.

28. The method of claim 27, wherein the autoimmune disease is selected from the group consisting of epidermolysis bullosa acquisita, pemphigus vulgaris, and Goodpasture syndrome, or a drug allergy.

29. The method of claim 27, wherein the anti-αβ TCR antibody or antigen binding fragment is selected from the group consisting of T10B9, MEDT-500, and TOL101.

30. The method of claim 27, wherein the anti-αβ TCR antibody or antigen binding fragment thereof is administered in an amount of 7 mg/day, 14 mg/day, 21 mg/day, 28 mg/day, 30 mg/day, 32 mg/day, 34 mg/day, 35 mg/day, 36 mg/day, 38 mg/day, 40 mg/day, 42 mg/day, 44 mg/day, 46 mg/day, 48 mg/day, 50 mg/day, 52 mg/day, 54 mg/day, 56 mg/day or 58 mg/day, or combinations thereof.

31. The method of claim 27, wherein the anti-αβ TCR antibody or antigen binding fragment thereof is administered according to a dosing schedule comprising: 14 mg at day 1, 21 mg at day 2, 28 mg at day 3, 42 mg at day 4, 42 mg at day 5, and 42 mg at day 6.

32. (canceled)

33. The method of claim 27, wherein the immune mediated adverse drug reaction is delayed exanthema without systemic symptoms (maculopapular emption), contact dermatitis, drug-induced hypersensitivity syndrome/drug reaction with eosinophilia and systemic symptoms (DRESS)/hypersensitivity syndrome, Stevens-Johnson syndrome (SJS)/toxic epidermal necrolysis (TEN), acute generalized exanthematous pustulosis, fixed drug eruption, and single organ involvement pathologies, such as drug-induced liver injury and pancreatitis, delayed exanthema without systemic symptoms (maculopapular eruption), contact dermatitis, drug-induced hypersensitivity syndrome/drug reaction with eosinophilia and systemic symptoms (DRESS)/hypersensitivity syndrome, Stevens-Johnson syndrome (SJS)/toxic epidermal necrolysis (TEN), acute generalized exanthematous pustulosis, fixed drug eruption, and single organ involvement pathologies, such as drug-induced liver injury and pancreatitis.

34. (canceled)

35. The method of claim 1, wherein the anti-αβ TCR antibody or antigen binding fragment at least one of: reduces CD3+ T cell counts to <25 T cell/mm³, reduces CD3+ T cell counts to 50%, 75% or 90% of baseline, allows for a CD3+ T cell rate of recovery of 7, 15 or 30 days, results in an anti-drug response of <20%, permits once daily dosing, does not suppress or delete γδ T-cells, does not suppress or delete B-cells and other white blood cells, does not suppress or delete platelets, or exhibits a reduced immune activation potential.

36.-43. (canceled)

44. The method of claim 1, wherein the activity of the anti-αβ TCR antibody or antigen binding fragment thereof is mimicked by a chemical entity, peptide or RNA/DNA-based molecule.

45. The method of claim 24, wherein the activity of TOL101, T10B9 or MEDI-500 is mimicked by a chemical entity, peptide or RNA/DNA-based molecule.

46. The method of claim 1, wherein the IM-ADR occurs subsequent to administration one or more checkpoint inhibitors to the subject or wherein the IM-ADR is SJS/TEN.

47. (canceled)

48. The method of claim 46, wherein the one or more checkpoint inhibitors is selected from ipilimumab, nivolumab, pembrolizumab, and atezolizumab, or a combination thereof.

49. A method of treating subject having cancer with one or more checkpoint inhibitors in combination with an anti-αβ T cell receptor (TCR) antibody or an antigen binding fragment thereof, wherein the one or more checkpoint inhibitors and the anti-αβ T cell receptor (TCR) antibody or an antigen binding fragment thereof are administered to the subject concurrently or sequentially.

50. (canceled)

51. The method of claim 49, wherein the one or more checkpoint inhibitors is selected from ipilimumab, nivolumab, pembrolizumab, and atezolizumab, or a combination thereof.

52. (canceled)

53. The method of claim 49, wherein the anti-αβ TCR antibody or antigen binding fragment is selected from the group consisting of T10B9, MEDI-500, and TOL101.


SEQUENCE LISTING (9 sequences)

SEQ ID NO: 1 - protein, 207 aa, Homo sapiens
MAKTTQPNSMESNEEEPVHLPCNHSTISGTDYIHWYRQLPSQGPEYVIHGLTSNVNNRMASLAIAEDRKSSTLILHRATLRDAAVYYCILPLAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 2 - protein, 256 aa, Homo sapiens
MAGSHMGVSQSPRYKVAKRGQDVALRCDPISGHVSLFWYQQALGQGPEFLTYFQNEAQLDKSGLPSDRFFAERPEGSVSTLKIQRTQQEDSAVYLCASSLGQAYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCTSGDDDDK

SEQ ID NO: 3 - DNA, 708 bp, Mus. sp.
atggattttcaagtgcagattttcagcttcctgctaatcagtgcctcagtcataatatccagaggacaaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 4 - DNA, 1782 bp, Mus sp.
atggaaaggcactggatctttctactcctgttgtcagtaactgcaggtgtccactcccaggtccagctgcagcagtctggggctgaactggcaagacctggggcctcagtgaagatgtcctgcaaggcttctggctacacctttactagctacacgatgcactgggtaaaacagaggcctggacagggtctggaatggattggatacattaatcctagcagtggttatactaattacaatcagaagttcaaggacaaggccacattgactgcagacaaatcctccagcacagcctacatgcaactgagcagcctgacatctgaggactctgcagtctattactgtgcaagatggagggacgcgtactatgctatggactactggggtcaaggaacctcagtcaccgtctcctcagagagtcagtccttcccaaatgtcttccccctcgtctcctgcgagagccccctgtctgataagaatctggtggccatgggctgcctggcccgggacttcctgcccagcaccatttccttcacctggaactaccagaacaacactgaagtcatccagggtatcagaaccttcccaacactgaggacagggggcaagtacctagccacctcgcaggtgttgctgtctcccaagagcatccttgaaggttcagatgaatacctggtatgcaaaatccactacggaggcaaaaacagagatctgcatgtgcccattccagctgtcgcagagatgaaccccaatgtaaatgtgttcgtcccaccacgggatggcttctctggccctgcaccacgcaagtctaaactcatctgcgaggccacgaacttcactccaaaaccgatcacagtatcctggctaaaggatgggaagctcgtggaatctggcttcaccacagatccggtgaccatcgagaacaaaggatccacaccccaaacctacaaggtcataagcacacttaccatctctgaaatcgactggctgaacctgaatgtgtacacctgccgtgtggatcacaggggtctcaccttcttgaagaacgtgtcctccacatgtgctgccagtccctccacagacatcctaaccttcaccatccccccctcctttgccgacatcttcctcagcaagtccgctaacctgacctgtctggtctcaaacctggcaacctatgaaaccctgaatatctcctgggcttctcaaagtggtgaaccactggaaaccaaaattaaaatcatggaaagccatcccaatggcaccttcagtgctaagggtgtggctagtgtttgtgtggaagactggaataacaggaaggaatttgtgtgtactgtgactcacagggatctgccttcaccacagaagaaattcatctcaaaacccaatgaggtgcacaaacatccacctgctgtgtacctgctgccaccagctcgtgagcaactgaacctgagggagtcagccacagtcacctgcctggtgaagggcttctctcctgcagacatcagtgtgcagtggcttcagagagggcaactcttgccccaagagaagtatgtgaccagtgccccgatgccagagcctggggccccaggcttctactttacccacagcatcctgactgtgacagaggaggaatggaactccggagagacctatacctgtgttgtaggccacgaggccctgccacacctggtgaccgagaggaccgtggacaagtccactggtaaacccacactgtacaatgtctccctgatcatgtctgacacaggcggcacctgctattga

SEQ ID NO: 5 - DNA, 480 bp, Mus sp.
atgaagacccacctgcttctctggggagtcctggccatttttgttaaggctgtccttgtaacaggtgacgacgaagcgaccattcttgctgacaacaaatgcatgtgtacccgagttacctctaggatcatcccttccaccgaggatcctaatgaggacattgtggagagaaatatccgaattgttgtccctttgaacaacagggagaatatctctgatcccacctccccactgagaaggaactttgtataccatttgtcagacgtctgtaagaaatgcgatcctgtggaagtggagctggaagatcaggttgttactgccacccagagcaacatctgcaatgaagacgatggtgttcctgagacctgctacatgtatgacagaaacaagtgctataccactatggtcccacttaggtatcatggtgagaccaaaatggtgcaagcagccttgacccccgattcttgctaccctgactag

SEQ ID NO: 6 - protein, 235 aa, Mus sp.
MDFQVQIFSFLLISASVIISRGQIVLTQSPAIMSASPGEKVTMTCSASSSVSYMHWYQQKSGTSPKRWIYDTSKLASGVPARFSGSGSGTSYSLTISSMEAEDAATYYCQQWSSNPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 7 - protein, 593 aa, Mus sp.
MERHWIFLLLLSVTAGVHSQVQLQQSGAELARPGASVKMSCKASGYTFTSYTMHWVKQRPGQGLEWIGYINPSSGYTNYNQKFKDKATLTADKSSSTAYMQLSSLTSEDSAVYYCARWRDAYYAMDYWGQGTSVTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 8 - protein, 159 aa, Mus sp.
MKTHLLLWGVLAIFVKAVLVTGDDEATILADNKCMCTRVTSRIIPSTEDPNEDIVERNIRIVVPLNNRENISDPTSPLRRNFVYHLSDVCKKCDPVEVELEDQVVTATQSNICNEDDGVPETCYMYDRNKCYTTMVPLRYHGETKMVQAALTPDSCYPD

SEQ ID NO: 9 - protein, 253 aa, Artificial Sequence
  TOL101 ScFV
QVQLQQSGAELARPGASVKMSCKASGYTFTSYTMHWVKQRPGQGLEWIGYINPSSGYTNYNQKFKDKATLTADKSSSTAYMQLSSLTSEDSAVYYCARWRDAYYAMDYWGQGTSVTVSSGGGGSGGGGSGGGGSGGGGSQIVLTQSPAIMSASPGEKVTMTCSASSSVSYMHWYQQKSGTSPKRWIYDTSKLASGVPARFSGSGSGTSYSLTISSMEAEDAATYYCQQWSSNPFTFGSGTKLEIKRAHHHHHH